(19) United States
(12) Patent Application Publication
(10) Pub. No.: US 2026/0315402 A1
(43) Pub. Date: Oct. 15, 2026

(54) COMPOSITIONS AND METHODS FOR DELIVERY OF NUCLEIC ACIDS TO CELLS

(71) Applicant: TAKEDA PHARMACEUTICAL COMPANY LIMITED, Osaka (JP)

(72) Inventors: Mohammad Parvez Alam, Lexington, MA (US); Roshan Padmashali Manohara Padmashali, Lexington, MA (US)

(73) Assignee: TAKEDA PHARMACEUTICAL COMPANY LIMITED, Osaka (JP)

(21) Appl. No.: 19/483,715

(22) PCT Filed: May 15, 2024
(86) PCT No.: PCT/IB2024/000233

§ 371 Date: Nov. 12, 2025

Related U.S. Application Data
(60) Provisional application No. 63/466,639, filed on May 15, 2023.

(51) Int. Cl. A61K 9/51; A61K 9/00; A61K 31/711; A61K 47/18; A61K 47/22; A61K 47/24; A61K 47/26; A61K 47/28; A61P 3/00

(52) U.S. Cl. CPC A61K 9/5123; A61K 9/0019; A61K 9/5192; A61K 31/711; A61K 47/18; A61K 47/22; A61K 47/24; A61K 47/26; A61K 47/28; A61P 3/00

(57) ABSTRACT

The disclosure features novel lipid nanoparticle formulations and use thereof. Lipid nanoparticles include cationic ionizable lipids, helper lipids, sterols, and conjugated lipids. Lipid nanoparticles further including therapeutics and/or prophylactics such as DNA are useful in the delivery of therapeutics and/or prophylactics to cells or organs (e.g., mammalian cells or organs) to, for example, regulate polypeptide, protein, or gene expression.

### RELATED APPLICATIONS

The instant application claims priority to U.S. Provisional Application No. 63/466,639, filed May 15, 2023, the entire contents of which are expressly incorporated by reference herein.

### BACKGROUND

The effective targeted delivery of biologically active substances such as nucleic acids by lipid nanoparticle represents a continuing medical challenge. In particular, the delivery of nucleic acids to cells is made difficult by potential toxicity and instability. Thus, there exists a need to develop methods and compositions to facilitate the delivery of therapeutics and/or prophylactics such as nucleic acids to cells.

### SUMMARY OF THE INVENTION

Accordingly, in one aspect, the present invention provides a lipid nanoparticle (LNP) composition comprising: a. a cationic ionizable lipid; b. a helper lipid, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dimyristoyl-sn-glycero-phosphocholine (DMPC), and 1,2-dipalmitoyl-sn-glycero-3-phosphocholine (DPPC); c. a sterol, wherein the sterol is selected from the group consisting of cholesterol, β-sitosterol, campesterol, ergosterol, phytosterols (e.g., stigmastanol), and fucosterol; d. a conjugated-lipid, wherein the conjugated-lipid is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K and PEG-DMG-5000K; and e. a nucleic acid, wherein the nucleic acid is DNA and at least about 2 kilobases (kb), at least about 3 kb, at least about 4 kb, at least about 5 kb, at least about 6 kb, at least about 7 kb, at least about 8 kb, at least about 9 kb, or at least about 10 kb.

In one embodiment, the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA).

In one embodiment, the cationic ionizable lipid is DLin-MC3-DMA.

In one embodiment, the cationic ionizable lipid is DLin-K2C-DMA.

In one embodiment, the helper lipid is DOPC.

In one embodiment, the sterol is cholesterol.

In one embodiment, the molar ratio of the cationic ionizable lipid, the helper lipid, the sterol, and the conjugated lipid is about 20%-80%: 5%-20%: 36%-39%: 1%-4% (mol) respectively of the total lipid present in the LNP composition.

In one embodiment, the molar ratio of the cationic ionizable lipid, the helper lipid, the sterol, and the conjugated lipid is about 50%: 10%: 38.5%: 1.5% (mol) respectively of the total lipid present in the LNP composition.

In one embodiment, the cationic ionizable lipid is DLin-MC3-DMA, the helper lipid is DOPC, the sterol is cholesterol, and the conjugated lipid is PEG-DMG, and wherein the molar ratio of the cationic ionizable lipid, the helper lipid, the sterol, and the conjugated lipid is about 50%: 10%: 38.5%: 1.5% (mol) respectively of the total lipid present in the LNP composition.

In one embodiment, the cationic ionizable lipid is DLin-KC2-DMA, the helper lipid is DOPC, the sterol is cholesterol, and the conjugated lipid is PEG-DMG, and wherein the molar ratio of the cationic ionizable lipid, the helper lipid, the sterol, and the conjugated lipid is about 50%: 10%: 38.5%: 1.5% (mol) respectively of the total lipid present in the LNP composition.

In one embodiment, the LNP has a negative zeta potential.

In one embodiment, the LNP has a zeta potential between about −0.1 mV to about −15 mV, about −1.0 mV to about −10 mV, about −2.0 mV to about −8.0 mV, about −4.0 mV to about −6.0 mV, or about −5.0 mV to about −10 mV.

In one embodiment, the LNP has a zeta potential of at least-0.1 mV, at least-0.5 mV, at least-1.0 mV, at least-2.0 mV, at least-5.0 mV, at least-10 mV, or at least-15 mV.

In one embodiment, the LNP has a zeta potential of at most-0.1 mV, at most-0.5 mV, at most-1.0 mV, at most-2.0 mV, at most-5.0 mV, at most-10 mV, or at most-15 mV.

In one embodiment, the LNP composition has a polydispersity index (PDI) of less than 1.0, less than 0.9, less than 0.8, less than 0.7, less than 0.6, less than 0.5, less than 0.4, less than 0.3, less than 0.2, or less than 0.1.

In one embodiment, the LNP composition has a nitrogen/phosphate (N/P) ratio is about 2, about 3, about 4, about 5, about 6, about 7, about 8, about 9, about 10, or about 15. In one embodiment, the N/P ratio is about 4.

In another aspect, the present invention provides a pharmaceutical composition, comprising the LNP composition as disclosed herein, and a pharmaceutically acceptable carrier.

In one embodiment, the pharmaceutical composition further comprises a cryoprotectant.

In one embodiment, the cryoprotectant is a sugar, starch, polyvinyl pyrrolidone, polyethylene oxide, or a combination thereof.

In one embodiment, the pharmaceutical composition comprises a sugar.

In one embodiment, the sugar is a disaccharide.

In one embodiment, the sugar is selected from the group consisting of lactose, sucrose, trehalose, and a combination thereof.

In one embodiment, amount of sugar is between about 0% w/v and about 40% w/v. In one embodiment, the amount of sugar is at least 5% w/v, at least 10% w/v, at least 20% w/v, or at least 30%. In one embodiment, the amount of sugar is about 20% w/v.

In another aspect, the present invention provides a method of producing a pharmaceutical composition, comprising a) producing a lipid nanoparticle (LNP) by mixing a lipid mixture and an aqueous solution, wherein the lipid mixture comprises: i. a cationic ionizable lipid; ii. a helper lipid, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dimyristoyl-sn-glycero-phosphocholine (DMPC), and 1,2-dipalmitoyl-sn-glycero-3-phosphocholine (DPPC); iii. a sterol, wherein the sterol is selected from the group consisting of cholesterol, β-sitosterol, campesterol, ergosterol, phytosterols (e.g., stigmastanol), and fucosterol; iv. a conjugated lipid, wherein the conjugated lipid is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K and PEG-DMG-5000K; and the aqueous solution comprises: iv. a nucleic acid, wherein the nucleic acid is DNA and at least about 2 kilobases (kb), at least about 3 kb, at least about 4 kb, at least about 5 kb, at least about 6 kb, at least about 7 kb, at least about 8 kb, at least about 9 kb, or at least about 10 kb; and b) mixing the LNPs produced in step a) and a pharmaceutically acceptable carrier.

In one embodiment, the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA).

In one embodiment, the cationic ionizable lipid is DLin-MC3-DMA.

In one embodiment, the cationic ionizable lipid is DLin-KC2-DMA.

In one embodiment, the helper lipid is DOPC.

In one embodiment, the sterol is cholesterol.

In one embodiment, the pharmaceutical composition comprises a sugar.

In one embodiment, the molar ratio of the cationic ionizable lipid, the helper lipid, the sterol, and the conjugated lipid is about 20%-80%: 5%-20%: 36%-39%: 1%-4% (mol) of the total lipid present in the LNP composition.

In one embodiment, the molar ratio of the cationic ionizable lipid, the helper lipid, the sterol, and the conjugated lipid is about 50%: 10%: 38.5%: 1.5% (mol) of the total lipid present in the LNP composition.

In one embodiment, the molar ratio of the cationic ionizable lipid, the helper lipid, the sterol, and the conjugated lipid is about 50%: 10%: 38.5%: 1.5% (mol) of the total lipid present in the LNP composition.

In one embodiment, the cationic ionizable lipid is DLin-MC3-DMA, the helper lipid is DOPC, the sterol is cholesterol, and the conjugated lipid is PEG-DMG-2000K, and wherein the molar ratio of the cationic ionizable lipid, the helper lipid, the sterol, and the conjugated lipid is about 50%: 10%: 38.5%: 1.5% (mol) of the total lipid present in the LNP composition.

In one embodiment, the cationic ionizable lipid is DLin-KC2-DMA, the helper lipid is DOPC, the sterol is cholesterol, and the conjugated lipid is PEG-DMG-2000K, and wherein the molar ratio of the cationic ionizable lipid, the helper lipid, the sterol, and the conjugated lipid is about 50%: 10%: 38.5%: 1.5% (mol) of the total lipid present in the LNP composition.

In another aspect, the present invention provides a method of treating a disease or disorder, the method comprising administering the LNP composition as disclosed herein, the pharmaceutical composition as disclosed herein, or the pharmaceutical composition produced by the method as disclosed herein to a subject in need thereof.

In one embodiment, the disease or disorder is a lysosomal storage disease, an inborn errors of metabolism (IEM) or a hematological disorder.

In one embodiment, the LNP composition as disclosed herein, the pharmaceutical composition as disclosed herein, or the pharmaceutical composition produced by the method as disclosed herein is administered intravenously.

In one embodiment, the LNP composition of as disclosed herein, the pharmaceutical composition as disclosed herein, or the pharmaceutical composition produced by the method as disclosed herein is administered subcutaneously or intramuscularly.

In another aspect, the present invention provides a method of optimizing a Lipid nanoparticle (LNP) comprising steps of (1) screening LNPs in vitro and (2) screening LNPs in vivo using immune deficient animal, optionally repeating the step (1) and the step (2).

### BRIEF DESCRIPTION OF THE DRAWINGS

FIGS. 1A-1C. Physico-Chemical Properties of LNP-npDNA (ATP7B) Containing MC3/Lipid1/KC2 Ionizable Lipids in Huh7 Cells. FIG. 1A depicts LNP-npDNA (ATP7B) physio-chemical properties including particle size and polydispersity index (PDI). FIG. 1B depicts dependence of particle size (bar) and encapsulation efficiency (star symbol) on N/P ratio and ionizable lipids. FIG. 1C depicts dependence of zeta potential on N/P ratio and ionizable lipids.

FIGS. 2A-2B. Impact of LNP-npDNA Formulations at Different N/P Ratio on Transgene Expression in Huh7 Cells. FIG. 2A depicts percentage of viable cells as function of N/P ratio following incubation with LNP-npDNA (CAG-Luc) containing MC3 as ionizable-lipid. FIG. 2B depicts luciferase expression as function of N/P ratio following incubation with LNP-npDNA (CAG-Luc) containing MC3 as ionizable-lipid.

FIGS. 3A-3C Impact of LNP-npDNA Formulations Containing Varying Ionizable Lipids at Different N/P Ratio on Transgene Expression in Huh7 Cells. FIG. 3A depicts percentage of viable cells as function of N/P ratio following incubation with LNP-npDNA (ATP7B) containing ionizable-lipids shown in legend. FIG. 3B depicts quantification of DNA1 expression as function of N/P ratio following incubation with LNP-npDNA (ATP7B) containing ionizable-lipids shown in legend. FIG. 3C depicts DNA1 expression as function of N/P ratio following incubation with LNP-npDNA (ATP7B) containing ionizable-lipids shown in FIG. 3B. Treatment of Huh7 cells were performed using 50 μg/mL stock instead of 25 μg/mL stock for other groups.

FIGS. 4A-4C Physico-Chemical Properties and Cell Viability of LNP-npDNA (ATP7B) Systems Containing MC3 and Different Helper Lipids in Huh7 Cells. FIG. 4A depicts LNP-npDNA (ATP7B) physico-chemical properties, including particle size and PDI. FIG. 4B depicts dependence of particle size (bar) and encapsulation efficiency (star symbol) on N/P ratio and helper lipids. FIG. 4C depicts cell viability as function of on N/P ratio and helper lipids.

FIG. 5 depicts In-vitro hATP7B protein expression.

FIGS. 6A-6B. FIG. 6A LNP-npDNA (CAG-Luc) System Containing Lipid 1 Ionizable Lipid Exhibit Cell Viability and Transfection Dose Dependence in Huh7 Cells. FIG. 6A depicts cell viability dose response. FIG. 6B depicts impact of LNP-npDNA (CAG-Luc) dose on transgene expression.

FIGS. 7A-7C. Amino Lipid Comparison with Optimal Helper Lipid/(N/P) Combination. FIG. 7A depicts in vitro cell viability with different LNP formulations and payloads LSP₁-hATP7b (wt), LSP₁-hATP7b (CodOpt), LSP₁-hATP7b (Trunc), or LSP₂-eGFP (wt). FIG. 7B depicts in vitro protein expression of formulations depicted in FIG. 7A. FIG. 7C depicts in vitro eGFP with LNP formulations including DOPC.

FIG. 8. depicts in vivo evaluation of DOPC containing LNP-DNA formulations with ATP7B and eGFP payload nanoplasmids.

FIGS. 9A-9F. in vivo evaluation of DOPC containing LNP-DNA formulations with ATP7B and eGFP payload nanoplasmids in mice. Day 7 post dose: npMY159 (hATP7B trunc) at 1.3 mg/kg+npOTS006 (eGFP) at 0.7 mg/kg. FIG. 9A depicts protein expression of hATP7B; FIG. 9B depicts the protein levels of hATP7B; FIG. 9C depicts mRNA levels of hATP7B; FIG. 9D depicts npDNA levels. FIG. 9E depicts eGFP protein levels and FIG. 9F depicts eGFP mRNA levels.

FIG. 10 depicts in vivo evaluation in immune deficient mice of DOPC containing LNP-DNA formulations with multiple nanoplasmid constructs of varying transgene sizes and different promoter sequences.

FIGS. 11A-11B depicts substantial presence of npDNA (FIG. 11A) and mRNA (FIG. 11B) copies from delivered constructs using Lipid1-DOPC formulations in mice at Day 7 and 28 post dose.

FIGS. 12A-12D. Large transgene expression delivered via LNP formulations in mice. FIG. 12A depicts western blot of hATP7B (approx. 1450 aa in size) expression and quantification at day 7. FIG. 12B depicts quantification of FVII-BDD expression in immune deficient (ID) mice. FIG. 12C depicts expression of eGFP in mice. FIG. 12D depicts eGFP expression shown by immunohistochemistry.

FIGS. 13A-13D. Small transgene expression (expressed proteins approx. 350-450 amino acids in length) delivered via LNP formulations in mice. FIG. 13A depicts expression of PAH under the control of promoters prTTR or prCB shown by immunohistochemistry. FIG. 13B depicts quantification of protein expression of OTC under the control of prTTR. FIG. 13C depicts expression of OTC shown by immunohistochemistry. FIG. 13D depicts quantification of protein expression of GLA over 28 days.

FIG. 14 depicts protein expression of ATP7B, PAH, eGFP, OTC, or hFVIII in treated mice shown by immunohistochemistry.

FIGS. 15A-15D. FIG. 15A depicts protein expression of ATP7B in liver in wt mice compared to ATP7B expression levels in human liver. FIG. 15B depicts the quantification of protein expression levels shown in FIG. 15A. FIG. 15C depicts expression of GLA in wt mice compared to the saline control. FIG. 15D depicts expression of FVIII-BODD-SQ in wt and immune deficient (ID) mice.

FIG. 16 depicts the scheme of optimizing and screening LNP.

### DETAILED DESCRIPTION

### Definitions

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

The terms “purify,” “purified,” “purification” means to make substantially pure or clear from unwanted components, material defilement, admixture or imperfection.

The terms “significant” or “significantly” are used synonymously with the term “substantially.” The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “stable” refers to a compound that is sufficiently robust to survive isolation to a useful degree of purity from a reaction mixture, and preferably capable of formulation into an efficacious therapeutic agent. The terms “stabilize” and “stabilized,” means to make or become stable.

### Lipid Nanoparticle Formulation

The present invention provides new compositions of matter, comprising a lipid nanoparticle comprising a. a cationic ionizable lipid, wherein the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA); b. a helper lipid, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dimyristoyl-sn-glycero-phosphocholine (DMPC), and 1,2-dipalmitoyl-sn-glycero-3-phosphocholine (DPPC); c. a sterol, wherein the sterol is selected from the group consisting of cholesterol, β-sitosterol, campesterol, ergosterol, phytosterols (e.g., stigmastanol), and fucosterol; d. a conjugated lipid, wherein the conjugated lipid is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K, and PEG-DMG-5000K; and e. a nucleic acid, wherein the nucleic acid is DNA and at least 2 kilobases (kb), at least 3 kb, at least 4 kb, at least 5 kb, at least 5 kb, at least 7 kb, at least 8 kb, at least 9 kb, or at least 10 kb.

As used herein, the term “lipid nanoparticle” or “LNP” refers to a structure comprising an internal lipophilic core surrounded by a hydrophilic phase encapsulating the core. The ionic interaction resulting from the different lipophilic and hydrophilic components of the nanoparticle generates independent and observable physical characteristics, the mean size of which is equal to or less than 1 μm, i.e., a mean size comprised between 1 and 1000 nm. LNP may comprise a cationic ionizable lipid, a helper lipid, a sterol and conjugated lipid as a lipid.

“Average size” is understood as the average diameter of the population of nanoparticles comprising the lipophilic phase and the hydrophilic phase. The mean size of these systems can be measured by standard methods known by the person skilled in the art, and which are described, for example, in the experimental part below.

The terms “ionizable lipid” or “cationic ionizable lipid” and “amino lipids” are used herein interchangeably and refer to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. The cationic ionizable lipid used in the lipid nanoparticle is not particularly limited. The cationic ionizable lipid can be selected appropriately from the cationic ionizable lipids known by the person skilled in the art. For example, cationic lipids described in WO 2015/011633, WO 2016/021683, WO 2011/153493, WO 2013/126803, WO 2010/054401, WO 2010/042877, WO 2016/104580, WO 2015/005253, WO 2014/007398, WO 2017/117528, WO 2017/075531, WO 2017/00414, WO 2015/199952, US 2015/0239834, WO 2019/131839, or WO 2020/032184 can be in the lipid nanoparticles. In some embodiments, the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA). In some embodiments, the cationic ionizable lipid is DLin-MC3-DMA. In some embodiments, the cationic ionizable lipid is DLin-KC2-DMA.

The term “helper lipid”, also known as “neutral lipid”, refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived. In some embodiments, the helper lipid is DOPC.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, β-sitosterol, campesterol, fucosterol, ergosterol, phytosterols (e.g., stigmastanol), fecosterol, ergosterol, bassicasterol, tomatidine, tomatine, ursolic, alpha-tocopherol, and mixtures thereof. In certain embodiments, the structural lipid is cholesterol.

As used herein, the term “conjugated lipid” refers to a lipid molecule conjugated with a non-lipid molecule, such as a PEG (e.g., PEG-lipid), polyoxazoline, polyamide, or polymer (e g., cationic polymer). In some embodiments, the conjugated lipid is a PEG-lipid and is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K, and PEG-DMG-5000K. In some embodiments, the conjugated lipid is PEG-DMG-2000K.

In some embodiments, the cationic ionizable lipid is between about 20% to about 80%, about 25% and about 80%, about 30% and about 80%, about 35% and about 80%, about 40% and about 80%, about 45% and about 80%, about 50% and 80%, about 55% and about 80%, about 60% and about 80%, about 65% and about 80%, about 70% and about 80%, about 75% and about 80%, about 20% to about 75%, about 25% and about 75%, about 30% and about 75%, about 35% and about 75%, about 40% and about 75%, about 45% and about 75%, about 50% and 75%, about 55% and about 75%, about 60% and about 75%, about 65% and about 75%, about 70% and about 75%, about 20% to about 70%, about 25% and about 70%, about 30% and about 70%, about 35% and about 70%, about 40% and about 70%, about 45% and about 70%, about 50% and 70%, about 55% and about 70%, about 60% and about 70%, about 65% and about 70%, about 20% to about 65%, about 25% and about 65%, about 30% and about 65%, about 35% and about 65%, about 40% and about 65%, about 45% and about 65%, about 50% and 65%, about 55% and about 65%, about 60% and about 65%, about 20% to about 60%, about 25% and about 60%, about 30% and about 60%, about 35% and about 60%, about 40% and about 60%, about 45% and about 60%, about 50% and 60%, about 55% and about 60%, about 20% to about 55%, about 25% and about 55%, about 30% and about 55%, about 35% and about 55%, about 40% and about 55%, about 45% and about 55%, about 50% and 60%, about 20% to about 50%, about 25% and about 50%, about 30% and about 50%, about 35% and about 50%, about 40% and about 50%, about 45% and about 50%, about 20% to about 45%, about 25% and about 45%, about 30% and about 45%, about 35% and about 45%, about 40% and about 45%, about 20% to about 40%, about 25% and about 40%, about 30% and about 40%, about 35% and about 40%, about 20% to about 35%, about 25% and about 35%, about 30% and about 35%, about 20% to about 30%, about 25% and about 30%, or about 20% and about 25% of the total lipid in the lipid nanoparticle. In some embodiments, the cationic ionizable lipid is about 20%, about 25%, about 30%, about 35%, about 40%, about 45%, about 50%, about 55%, about 60%, about 65%, about 70%, about 75%, about 80% of the total lipid in the lipid nanoparticle. In some embodiments, the cationic ionizable lipid is at least 20%, at least 25%, at least 30%, at least 35%, at least 40%, at least 45%, at least 50%, at least 55%, at least 60%, at least 65%, at least 70%, at least 75%, at least 80% of the total lipid in the lipid nanoparticle.

In some embodiments, the helper lipid is between about 5% and about 20%, about 10% and about 20%, about 15% and about 20%, about 5% and about 15%, about 10% and about 15%, or about 5% and about 10% of the total lipid in the lipid nanoparticle. In some embodiments, the helper lipid is about 5%, about 10%, about 15%, or about 20% of the total lipid in the lipid nanoparticle. In some embodiments, the helper lipid is at least 5%, at least 10%, at least 15%, or at least 20% of the total lipid in the lipid nanoparticle.

In some embodiments, the sterol is between about 36% and about 39%, about 36% and 38.5%, about 36% and about 38%, about 36% and about 37.5%, about 36% and about 37%, about 36% and about 36.5%, about 36.5% and about 39%, about 36.5% and 38.5%, about 36.5% and about 38%, about 36.5% and about 37.5%, about 36.5% and about 37%, about 37% and about 39%, about 37% and 38.5%, about 37% and about 38%, about 37% and about 37.5%, about 37.5% and about 39%, about 37.5% and 38.5%, about 37.5% and about 38%, about 38% and about 39%, about 38% and 38.5%, or about 38.5% and about 39% of the total lipid in the lipid nanoparticle. In some embodiments, the sterol is about 36%, about 36.5%, about 37%, about 37.5%, about 38%, about 38.5%, or about 39% of the total lipid in the lipid nanoparticle. In some embodiments, the sterol is at least 36%, at least 36.5%, at least 37%, at least 37.5%, at least 38%, at least 38.5%, or at least 39% of the total lipid in the lipid nanoparticle.

In some embodiments, the conjugated lipid is between about 1.0% and about 4.0%, about 1.0% and about 3.5%, about 1.0% and about 3.0%, about 1.0% and about 2.5%, about 1.0% and about 2.0%, about 1.0% and about 1.5%, about 1.5% and about 4.0%, about 1.5% and about 3.5%, about 1.5% and about 3.0%, about 1.5% and about 2.5%, about 1.5% and about 2.0%, about 2.0% and about 4.0%, about 2.0% and about 3.5%, about 2.0% and about 3.0%, about 2.0% and about 2.5%, about 2.5% and about 4.0%, about 2.5% and about 3.5%, about 2.5% and about 3.0%, about 3.0% and about 4.0%, about 3.0% and about 3.5%, or about 3.5% and about 4.0% of the total lipid in the lipid nanoparticle. In some embodiments, the conjugated lipid is about 1.0%, about 1.5%, about 2.0%, about 2.5%, about 3.0%, about 3.5%, or about 4.0% of the total lipid in the lipid nanoparticle. In some embodiments, the conjugated lipid is at least 1.0%, at least 1.5%, at least 2.0%, at least 2.5%, at least 3.0%, at least 3.5%, or at least 4.0% of the total lipid in the lipid nanoparticle.

In some embodiments, the molar ratio of the cationic ionizable lipid, the helper lipid, the sterol, and the conjugated lipid is about 20%-80%: 5%-20%: 36%-39%: 1%-4% (mol) of the total lipid present in the LNP composition.

In some embodiments, the molar ratio of the cationic ionizable lipid, the helper lipid, the sterol, and the conjugated lipid is about 50%: 10%: 38.5%: 1.5% (mol) of the total lipid present in the LNP composition.

In some embodiments, the cationic ionizable lipid is DLin-MC3-DMA, the helper lipid is DOPC, the sterol is cholesterol, and the conjugated lipid is PEG-DMG, and wherein the molar ratio of the cationic ionizable lipid, the helper lipid, the sterol, and the conjugated lipid is about 50%: 10%: 38.5%: 1.5% (mol) of the total lipid present in the LNP composition.

In some embodiments, the cationic ionizable lipid is DLin-KC2-DMA, the helper lipid is DOPC, the sterol is cholesterol, and the conjugated lipid is PEG-DMG, and wherein the molar ratio of the cationic ionizable lipid, the helper lipid, the sterol, and the conjugated lipid is about 50%: 10%: 38.5%: 1.5% (mol) of the total lipid present in the LNP composition.

In some embodiments, the composition comprises at least one lipid nanoparticle encapsulating the nucleic acid. In some embodiments, the lipid nanoparticle comprises a negative charge. In some embodiments, the lipid nanoparticle comprises a positive charge.

As used herein, the terms “aqueous solution” and “buffer” are interchangeable. In some embodiments, the aqueous solution is a buffer selected from the group consisting of sodium acetate buffer, HEPES (4-(2-hydroxyethyl)-1-piperazineethanesulfonic acid), PBS (phosphate buffered saline), TRIS (tris(hydroxymethyl)aminomethane), MES (2-(N-morpholino) ethanesulfonic acid), and citrate buffer. In one embodiment, the aqueous solution is sodium acetate buffer. In one embodiment, the aqueous solution is PBS.

In some embodiments, the aqueous solution has a pH of about pH 5.0 to about pH 8.0. In some embodiments, the pH is about 5.0.

According to the present invention, a lipid solution contains a mixture of lipids suitable to form lipid nanoparticles for encapsulation of nucleic acid (e.g., DNA or RNA). In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

As used herein, the phrase “N/P ratio” refers to the ratio of positively-chargeable lipid, lipidoid or polymer amine (N=nitrogen) groups to negatively-charged nucleic acid phosphate (P) groups, is perhaps one of the most important physicochemical properties of polymer-based gene delivery vehicles. In some embodiments, the nucleic acid is RNA. In some embodiments, the nucleic acid is DNA. In some embodiments, the N/P ratio is about 1 to about 35, about 1 to about 30, about 1 to about 25, about 1 to about 20, about 1 to about 15, about 1 to about 10, about 1 to about 5, about 5 to about 35, about 5 to about 30, about 5 to about 25, about 5 to about 20, about 5 to about 15, about 5 to about 10, about 10 to about 35, about 10 to about 30, about 10 to about 25, about 10 to about 20, about 10 to about 15, about 15 to about 35, about 15 to about 30, about 15 to about 25, about 15 to about 20, about 20 to about 35, about 20 to about 30, about 20 to about 25, about 25 to about 35, about 25 to about 30, or about 30 to about 35. Preferably, the nucleic acid is RNA and the N/P ratio is about 5 to about 30. In some embodiments, the nucleic acid is DNA and the N/P ratio is about 15 to about 20. In some embodiments, the nucleic acid is DNA and the N/P ratio is 4.

As used herein, the term “zeta potential” refers to the electrostatic potential of a molecule or particle such as a LNP, measured at the plane of hydrodynamic slippage outside the surface of the molecule or particle. Usually measured by electrophoretic mobility. Related to the surface potential and a measure of the electrostatic forces of repulsion the particle is likely to meet when encountering another of the same sign of charge. In some embodiments, the zeta potential of the lipid nanoparticle, with or without the polymer coating, can be measured.

In some embodiments, the LNP composition has a negative zeta potential. In some embodiments, the zeta potential is between about −0.1 mV to about −50 mV, about −0.5 mV to about −50 mV, about −1.0 mV to about −50 mV, about −2.0 mV to about −50 mV, about −5.0 mV to about −50 mV, about −10 mV to about −50 mV, about −15 mV to about −50 mV, about −20 mV to about −50 mV, about −25 mV to about −50 mV, about −30 mV to about −50 mV, about −35 mV to about −50 mV, about −40 mV to about −50 mV, about −45 mV to about −50 mV, about −0.1 mV to about −45 mV, about −0.5 mV to about −45 mV, about −1.0 mV to about −45 mV, about −2.0 mV to about −45 mV, about −5.0 mV to about −45 mV, about −10 mV to about −45 mV, about −15 mV to about −45 mV, about −20 mV to about −45 mV, about −25 mV to about −45 mV, about −30 mV to about −45 mV, about −35 mV to about −45 mV, about −40 mV to about −45 mV, about −0.1 mV to about −40 mV, about −0.5 mV to about −40 mV, about −1.0 mV to about −40 mV, about −2.0 mV to about −40 mV, about −5.0 mV to about −40 mV, about −10 mV to about −40 mV, about −15 mV to about −40 mV, about −20 mV to about −40 mV, about −25 mV to about −40 mV, about −30 mV to about −40 mV, about −35 mV to about −40 mV, about −0.1 mV to about −35 mV, about −0.5 mV to about −35 mV, about −1.0 mV to about −35 mV, about −2.0 mV to about −35 mV, about −5.0 mV to about −35 mV, about −10 mV to about −35 mV, about −15 mV to about −35 mV, about −20 mV to about −35 mV, about −25 mV to about −35 mV, about −30 mV to about −35 mV, about −0.1 mV to about −30 mV, about −0.5 mV to about −30 mV, about −1.0 mV to about −30 mV, about −2.0 mV to about −30 mV, about −5.0 mV to about −30 mV, about −10 mV to about −30 mV, about −15 mV to about −30 mV, about −20 mV to about −30 mV, about −25 mV to about −30 mV, about −0.1 mV to about −25 mV, about −0.5 mV to about −25 mV, about −1.0 mV to about −25 mV, about −2.0 m V to about −25 mV, about −5.0 mV to about −25 mV, about −10 mV to about −25 mV, about −15 mV to about −25 mV, about −20 mV to about −25 mV, about −0.1 mV to about −20 mV, about −0.5 mV to about −20 mV, about −1.0 mV to about −20 mV, about −2.0 mV to about −20 mV, about −5.0 mV to about −20 mV, about −10 mV to about −20 mV, about −15 mV to about −20 mV, about −0.1 mV to about −15 mV, about −0.5 mV to about −15 mV, about −1.0 mV to about −15 mV, about −2.0 mV to about −15 mV, about −5.0 mV to about −15 mV, about −10 mV to about −15 mV, about −0.1 mV to about −10 mV, about −0.5 mV to about −10 mV, about −1.0 mV to about −10 mV, about −2.0 mV to about −10 mV, about −5.0 mV to about −10 mV, about −0.1 mV to about −5.0 mV, about −0.5 mV to about −5.0 mV, about −1.0 mV to about −5.0 mV, about −2.0 mV to about −5.0 mV, about −0.1 mV to about −2.0 mV, about −0.5 mV to about −2.0 mV, about −1.0 mV to about −2.0, about −0.1 mV to about −1.0 mV, about −0.5 mV to about −1.0 mV, or about −0.1 mV to about −0.5. In some embodiments, the zeta potential is at least-0.1 mV, at least-0.5 mV, at least-1.0 mV, at least-2.0 mV, at least-5.0 mV, at least-10 mV, at least-15 mV, at least-20 mV, at least-25 mV, at least-30 mV, at least-35 mV, at least-40 mV, at least-45 mV, or at least-50 mV. In some embodiments, the zeta potential is at most-0.1 mV, at most-0.5 mV, at most-1.0 mV, at most-2.0 mV, at most −5.0 mV, at most-10 mV, at most-15 mV, at most-20 mV, at most-25 mV, at most-30 mV, at most-35 mV, at most-40 mV, at most-45 mV, or at most-50 mV.

As used herein, the term “polydispersity index (PDI)” refers to the standard deviation (o) of the particle diameter distribution divided by the mean particle diameter. PDI is used to estimate the average uniformity of a particle solution. For example, a small value, e.g., less than 0.3, indicates a narrow particle size distribution. For example, a larger PDI value corresponds to a larger size distribution in the particle sample.

In some embodiments, the polydispersity index of the LNPs in a composition is less than 1.0, less than 0.9, less than 0.8, less than 0.7, less than 0.6, less than 0.5, less than 0.4, less than 0.3, less than 0.2, less than 0.1. In some embodiments, the polydispersity index of the LNPs in a composition is between about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.0, about 0.8 to about 0.9, or about 0.9 to about 1.0.

In some embodiments, the formed lipid nanoparticles are in an aqueous solution. In some embodiments, the aqueous solution is a buffer selected from the group consisting of sodium acetate buffer, HEPES (4-(2-hydroxyethyl)-1-piperazineethanesulfonic acid), PBS (phosphate buffered saline), TRIS (tris(hydroxymethyl)aminomethane), MES (2-(N-morpholino) ethanesulfonic acid), and citrate buffer. In one embodiment, the aqueous solution is sodium acetate buffer. In one embodiment, the aqueous solution is PBS.

In some embodiments, the LNP composition further comprises a sugar (e.g., the LNPs are in an aqueous solution comprising sugar). In some embodiments, the sugar is selected from the group consisting of lactose, sucrose, trehalose, and a combination thereof.

In some embodiments, the amount of sugar in the LNP composition is between about 0% w/v and about 40% w/v. In one embodiment, the amount of sugar is between about 0.1% w/v and about 40% w/v, about 1% w/v to about 40% w/v, about 1% w/v to about 35% w/v, about 1% w/v to about 30% w/v, about 1% w/v to about 25% w/v, about 1% w/v to about 20% w/v, about 1% w/v to about 15% w/v, about 1% w/v to about 10% w/v, about 1% w/v to about 5% w/v, about 5% w/v to about 40% w/v, about 5% w/v to about 35% w/v, about 5% w/v to about 30% w/v, about 5% w/v to about 25% w/v, about 5% w/v to about 20% w/v, about 5% w/v to about 15% w/v, about 5% w/v to about 10% w/v, about 10% w/v to about 40% w/v, about 10% w/v to about 35% w/v, about 10% w/v to about 30% w/v, about 10% w/v to about 25% w/v, about 10% w/v to about 20% w/v, about 10% w/v to about 15% w/v, about 15% w/v to about 40% w/v, about 15% w/v to about 35% w/v, about 15% w/v to about 30% w/v, about 15% w/v to about 25% w/v, about 15% w/v to about 20% w/v, about 20% w/v to about 40% w/v, about 20% w/v to about 35% w/v, about 20% w/v to about 30% w/v, about 20% w/v to about 25% w/v, about 25% w/v to about 40% w/v, about 25% w/v to about 35% w/v, about 25% w/v to about 30% w/v, about 30% w/v to about 40% w/v, about 30% w/v to about 35% w/v, or about 35% w/v to about 40% w/v. In one embodiment, the amount of sugar is at least 5% w/v, at least 10% w/v, at least 20% w/v, or at least 30% w/v. In one embodiment, preferably the amount of sugar is about 20% w/v.

### Therapeutic Agents

As used herein, the term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents” or “active ingredients.” Such agents include, but are not limited to, cytotoxins, radioactive ions, chemotherapeutic agents, small molecule drugs, proteins, and nucleic acids.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA, circular RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

In one embodiment, a nucleic acid, wherein the nucleic acid is DNA and at least 2 kilobases (kb), at least 3 kb, at least 4 kb, at least 5 kb, at least 5 kb, at least 7 kb, at least 8 kb, at least 9 kb, or at least 10 kb. In one embodiment, the nucleic acid molecule is between about 10 nucleotides (nt) and 20 kilobases (kb), about 500 nt to about 20 kb, about 1 kb to about 20 kb, about 2 kb to about 20 kb, about 3 kb to about 20 kb, about 4 kb to about 20 kb, about 5 kb to about 20 kb, about 6 kb to about 20 kb, about 7 kb to about 20 kb, about 8 kb to about 20 kb, about 9 kb to about 20 kb, about 10 kb to about 20 kb, about 15 kb to about 20 kb, about 10 nt and 15 kb, about 500 nt to about 15 kb, about 1 kb to about 15 kb, about 2 kb to about 15 kb, about 3 kb to about 15 kb, about 4 kb to about 15 kb, about 5 kb to about 15 kb, about 6 kb to about 15 kb, about 7 kb to about 15 kb, about 8 kb to about 15 kb, about 9 kb to about 15 kb, about 10 kb to about 15 kb, about 10 nt and 10 kb, about 500 nt to about 10 kb, about 1 kb to about 10 kb, about 2 kb to about 10 kb, about 3 kb to about 10 kb, about 4 kb to about 10 kb, about 5 kb to about 10 kb, about 6 kb to about 10 kb, about 7 kb to about 10 kb, about 8 kb to about 10 kb, about 9 kb to about 10 kb, about 10 nt and 8 kb, about 500 nt to about 8 kb, about 1 kb to about 8 kb, about 2 kb to about 8 kb, about 3 kb to about 8 kb, about 4 kb to about 8 kb, about 5 kb to about 8 kb, about 6 kb to about 8 kb, about 7 kb to about 8 kb, about 10 nt and 6 kb, about 500 nt to about 6 kb, about 1 kb to about 6 kb, about 2 kb to about 6 kb, about 3 kb to about 6 kb, about 4 kb to about 6 kb, about 5 kb to about 6 kb, about 10 nt and 5 kb, about 500 nt to about 5 kb, about 1 kb to about 5 kb, about 2 kb to about 5 kb, about 3 kb to about 5 kb, about 4 kb to about 5 kb, about 10 nt and 4 kb, about 500 nt to about 4 kb, about 1 kb to about 4 kb, about 2 kb to about 4 kb, about 3 kb to about 4 kb, about 10 nt and 3 kb, about 500 nt to about 3 kb, about 1 kb to about 3 kb, about 2 kb to about 3 kb, about 10 nt and 2 kb, about 500 nt to about 2 kb, about 1 kb to about 2 kb, about 10 nt and 1 kb, about 500 nt to about 1 kb, or about 10 nt to about 500 nt. Preferably, the nucleic acid is DNA, e.g., viral vector or plasmid, and is about 3 kb to about 20 kb. Preferably, the nucleic acid is DNA and at least 2 kilobases (kb), at least 3 kb, at least 4 kb, at least 5 kb, at least 5 kb, at least 7 kb, at least 8 kb, at least 9 kb, or at least 10 kb.

The nucleotide analog may be any molecule obtained by modifying a ribonucleotide, deoxyribonucleotide, RNA, or DNA to improve nuclease resistance, to stabilize, to enhance affinity with a complementary-strand nucleic acid or to enhance cell permeability as compared with RNA or DNA, or for visualization. The nucleotide analog may be a naturally-occurring molecule or a non-natural molecule, and examples thereof include a sugar-modified nucleotide analog and a phosphodiester bond-modified nucleotide analog.

The sugar-modified nucleotide analog may be any one obtained by addition of or substitution with a substance having an arbitrary chemical structure for a part or the whole of the chemical structure of the sugar of a nucleotide. Specific examples thereof include a nucleotide analog substituted with 2′-O-methylribose, a nucleotide analog substituted with 2′-O-propylribose, a nucleotide analog substituted with 2′-methoxyethoxyribose, a nucleotide analog substituted with 2′-O-methoxyethylribose, a nucleotide analog substituted with 2′-O-[2-(guanidinium)ethyl]ribose, a nucleotide analog substituted with 2′-O-fluororibose, a crosslinked artificial nucleic acid provided with two cyclic structures by introducing a crosslinked structure to the sugar moiety (Bridged Nucleic Acid) (BNA), more specifically, a locked artificial nucleic acid in which the oxygen atom at position 2′ and the carbon atom at position 4′ are crosslinked via methylene (Locked Nucleic Acid) (LNA) and an ethylene-crosslinked artificial nucleic acid (Ethylene bridged nucleic acid) (ENA).

The phosphodiester bond-modified nucleotide analog may be any one obtained by addition of or substitution with an arbitrary chemical substance for a part or the whole of the chemical structure of the phosphodiester bond of a nucleotide. Specific examples thereof include a nucleotide analog substituted with a phosphorothioate bond and a nucleotide analog substituted with an N3′-P5′ phosphoramidate bond.

The nucleic acid derivative may be any molecule obtained by adding to a nucleic acid another chemical substance to improve nuclease resistance, to stabilize, to enhance affinity with a complementary-strand nucleic acid, or to enhance cell permeability as compared with the nucleic acid, or for visualization. Specific examples thereof include a derivative with 5′-polyamine added, a derivative with cholesterol added, a derivative with steroid added, a derivative with bile acid added, a derivative with vitamin added, a derivative with Cy5 added, a derivative with Cy3 added, a derivative with 6-FAM added, and a derivative with biotin added.

The nucleic acid in the present invention is not limited to a particular nucleic acid, and may be a nucleic acid, for example, for the purpose of ameliorating a disease, a symptom, a disorder, or sickness, and mitigating a disease, a symptom, a disorder, or pathological condition or preventing the onset thereof (herein, occasionally referred to as “treatment or the like of a disease”), or a nucleic acid for regulating expression of a desired protein that is useful for research, even though the protein does not contribute to treatment or the like of a disease.

Specific examples of the nucleic acid in the present invention include siRNAs, shRNAs, miRNAs, miRNA mimics, antisense nucleic acids, ribozymes, mRNAs, decoy nucleic acids, aptamers, plasmid DNAs, Cosmid DNAs, and BAC DNAs. The nucleic acid is preferably an RNA such as an siRNA and an mRNA or an analog or derivative obtained by artificially modifying an RNA.

In the present invention, the “siRNA” refers to a double-stranded RNA or relative thereof consisting of 10 to 30 nucleotides, preferably of 15 to 25 nucleotides, and including complementary sequences. The siRNA includes protruding nucleotides preferably of one to three nucleotides, more preferably of two nucleotides, at the 3′-end. The moiety of complementary sequences may be completely complementary or include non-complementary nucleotides, but preferably are completely complementary.

The siRNA in the present invention is not limited to a particular siRNA, and, for example, an siRNA for knockdown of gene expression against a disease-related gene may be used. A disease-related gene refers to any gene or polynucleotide generating a transcription or translation product at an abnormal level or in an abnormal form in cells derived from tissue of a patient, as compared with tissue or cells from a disease-free control. For the siRNA in the present invention, an siRNA to regulate expression of a desired protein useful for research may be used.

In the present invention, the “mRNA” refers to an RNA including a nucleotide sequence that can be translated into a protein. The mRNA in the present invention is not limited to a particular mRNA and may be any mRNA capable of expressing a desired protein in cells. The mRNA is preferably an mRNA useful for pharmaceutical applications (e.g., applications of disease treatment) and/or applications for research, and examples of such mRNAs include an mRNA to express a marker protein such as luciferase in cells.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “RNA” means a molecule comprising at least one ribonucleotide residue. By “ribonucleotide” is meant a nucleotide with a hydroxyl group at the 2′ position of a β-D-ribo-furanose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA.

In some embodiments the RNA is a self-replicating RNA. In some embodiments, the RNA is mRNA. In some embodiments, the RNA is siRNA. In some embodiments, the nucleic acid is from about 1000 nucleotides to about 13000 nucleotides in length.

The lipid nanoparticles of the disclosure also typically have a total lipid: nucleic acid (e.g., DNA or RNA) ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1.

In some embodiments, the lipid nanoparticles comprise a nucleic acid (e.g., DNA or RNA) that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the nucleic acid (e.g., RNA or DNA) encapsulated therein.

The encapsulated nucleic acid is calculated by the formula: [(total nucleic molecules-free nucleic molecules)/(total nucleic molecules)]×100%.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

### Disease and Disorders

The disease is not limited to a particular disease, and examples of the disease include diseases listed below. Contents in each “( )” are examples of the corresponding disease-related gene, except for the case that specific disease examples are listed. Another example of the nucleic acid in the present invention is a nucleic acid that regulates the expression level of any of those disease-related genes (or a protein encoded by the disease-related gene).
(1) Diseases of blood system [anaemia (CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, ASAT), bare lymphocyte syndrome (TAPBP, TPSN, TAP2, ABCB3, PSF2, RING11, MHC2TA, C2TA, RFX5), hemorrhagic diseases (TBXA2R, P2RX1, P2X1), factor H and factor H-like 1 deficiencies (HF1, CFH, HUS), factor V and factor VIII deficiencies (MCFD2), factor VII deficiency (F7), factor X deficiency (F10), factor XI deficiency (F11), factor XII deficiency (F12, HAF), factor XIIIA deficiency (F13A1, F13A), factor XIIIB deficiency (F13B), Fanconi's anaemia (FANCA, FACA, FAI, FA, FAA, FAAP95, FAAP90, F1134064, FANCB, FANCC, FACC, BRCA2, FANCD1, FANCD2, FANCD, FACD, FAD, FACE, FACE, FANCF, XRCC9, FANCG, BRIP1, BACH1, FANCJ, PHF9, FANCL, FANCM, KIAA1596), haemophagocytic lymphohistiocytosis (PRF1, HPLH2, UNC13D, MUNC13-4, HPLH3, HLH3, FHL3), haemophilia A (F8, F8C, HEMA), haemophilia B (F9, HEMB), haemorrhagic disorders (PI, ATT, F5), leucocyte deficiency (ITGB2, CD18, LCAMB, LAD, EIF2B1, EIF2BA, EIF2B2, EIF2B3, EIF2B5, LVWM, CACH, CLE, EIF2B4), sickle cell anaemia (HBB), thalassaemia (HBA2, HBB, HBD, LCRB, HBA1), and so on];
(2) inflammatory/immunologic diseases [AIDS (KIR3DL1, NKAT3, NKB1, AMB11, KIR3DS1, IFNG, CXCL12, SDF1), autoimmune lymphoproliferative syndrome (TNFRSF6, APT1, FAS, CD95, ALPS1A), combined immunodeficiency (IL2RG, SCIDX1, SCIDX, IMD4), HIV infection (CCL5, SCYA5, D17S135E, TCP228, IL10, CSIF, CMKBR2, CCR2, DMKBR5, CCCKR5, CCR5), immunodeficiency (CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, TACI), inflammation (IL10, IL-1, IL-13, IL-17, IL-23, CTLA4), severe combined immunodeficiency (JAK3, JAKL, DCLREIC, ATREMIS, SCIDA, RAG1, RAG2, ADA, PTPRC, CD45, LCA, IL7R, CD3D, T3D, IL2RG, SCIDX1, SCIDX, IMD4), rheumatoid arthritis, psoriasis, inflammatory bowel diseases (e.g., Crohn's disease, ulcerative colitis), Sjogren's syndrome, Behcet's disease, multiple sclerosis, systemic lupus erythematosus, lupus nephritis, discoid lupus erythematosus, Castleman's disease, ankylosing spondylitis, polymyositis, dermatomyositis, polyarteritis nodosa, mixed connective tissue disease, dermatosclerosis, lupus profundus, chronic thyroiditis, Graves' disease, autoimmune gastritis, type I and type II diabetes mellitus, autoimmune haemolytic anaemia, autoimmune neutropenia, thrombocytopenia, atopic dermatitis, chronic active hepatitis, myasthenia gravis, graft-versus-host disease, Addison's disease, abnormal immune response, arthritis, dermatitis, radiodermatitis, primary biliary cirrhosis, and so on];
(3) metabolic/liver/kidney diseases [amyloid neuropathy (TTR, PALB), amyloidosis (APOA1, APP, AAA, CVAP, AD1, GSN, FGA, LYZ, TTR, PALB), non-alcoholic steatohepatitis and hepatic fibrosis (COL1A1), cirrhosis (KRT18, KRT8, CIRHIA, NAIC, TEX292, KIAA1988), cystic fibrosis (CFTR, ABCC7, CF, MRP7), glycogen storage disease (SLC2A2, GLUT2, G6PC, G6PT, G6PT1, GAA, LAMP2, LAMPB, AGL, GDE, GBE1, GYS2, PYGL, PFKM), hepatocellular adenoma (TCF1, HFNIA, MODY3), hepatic failure (SCOD1, SCO1), hepatic lipase deficiency (LIPC), hepatoblastoma (CTNNB1, PDFGRL, PDGRL, PRLTS, AXIN1, AXIN, TP53, P53, LFS1, IGF2R, MPRI, MET, CASP8, MCH5), medullary cystic kidney disease (UMOD, HNFJ, FJHN, MCKD2, ADMCKD2), phenylketonuria (PAH, PKU1, QDPR, DHPR, PTS), multicystic kidney and liver diseases (FCYT, PKHD1, APRKD, PDK1, PDK2, PDK4, PDKTS, PRKCSH, G19P1, PCLD, SEC63), and so on];
(4) nervous system diseases [ALS (SOD1, ALS2, STEX, FUS, TARDBP, VEGF), Alzheimer's disease (APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIPIL, PTIP, A2M, BLMH, BMH, PSEN1, AD3), autism (BZRAP1, MDGA2, GLO1, MECP2, RTT, PPMX, MRX16, MRX79, NLGN3, NLGN4, KIAA1260, AUTSX2), fragile X syndrome (FMR2, FXR1, FXR2, mGLUR5), Huntington's disease (HD, IT15, PRNP, PRIP, JPH3, JP3, HDL2, TBP, SCA17), Parkinson's disease (NR4A2, NURR1, NOT, TINUR, SNCAIP, TBP, SCA17, SNCA, NACP, PARK1, PARK4, DJ1, DBH, NDUFV2), Rett syndrome (MECP2, RTT, PPMX, MRX16, MRX79, CDKL5, STK9), schizophrenia (GSK3,5-HTT, COMT, DRD, SLC6A3, DAOA, DTNBP1), secretase-related disorder (APH-1), and so on];
(5) eye diseases [age-related macular degeneration (Abcr, Cc12, cp, Timp3, cathepsin D, Vldlr, Ccr2), cataract (CRYAA, CRYA1, CRYBB2, CRYB2, PITX3, BFSP2, CP49, CP47, PAX6, AN2, MGDA, CRYBA1, CRYB1, CRYGC, CRYG3, CCL, LIM2, MP19, CRYGD, CRYG4, BSFP2, CP49, CP47, HSF4, CTM, MW, AQPO, CRYAB, CRYA2, CTPP2, CRYBB1, CRYGD, CRYG4, CRYA1, GJA8, CX50, CAE1, GJA3, CX46, CZP3, CAE3, CCM1, CAM, KRIT1), corneal opacity (APOA1, TGFB1, CSD2, CDGG1, CSD, BIGH3, CDG2, TASTD2, TROP2, MIS1, VSX1, RINX, PPCD, PPD, KTCN, COL8A2, FECD, PPCD2, PIP5K3, CFD), congenital hereditary corneal plana (KERA, CNA2), glaucoma (MYOC, TIGR, GLCIA, JOAG, GPOA, OPTN, GLCIE, FIP2, HYPL, NRP, CYP1B1, GLC3A, OPA1, NTG, NPG, CYP1B1, GLC3A), Leber's congenital amaurosis (CRB1, RP12, CRX, CORD2, CRD, RPGRIP1, LCA6, CORDS, RPE65, RP20, AIPL1, LCA4, GUCY2D, GUC2D, LCA1, CORD6, RDH12, LCA3), macular dystrophy (ELOVL4, ADMD, STGD2, STGD3, RDS, RP7, PRPH2, PRPH, AVMD, AOFMD, VMD2), and so on]; and
(6) neoplastic diseases [malignant tumor, neovascular glaucoma, infantile hemangioma, multiple myeloma, chronic sarcoma, metastatic melanoma, Kaposi's sarcoma, angioproliferation, cachexia, metastasis or the like of breast cancer, cancer (e.g., colorectal cancer (e.g., familial colorectal cancer, hereditary nonpolyposis colorectal cancer, gastrointestinal stromal tumor), lung cancer (e.g., non-small cell lung cancer, small cell lung cancer, malignant mesothelioma), mesothelioma, pancreatic cancer (e.g., pancreatic ductal carcinoma), gastric cancer (e.g., papillary adenocarcinoma, mucinous adenocarcinoma, adenosquamous cell carcinoma), breast cancer (e.g., invasive ductal breast carcinoma, noninvasive ductal breast carcinoma, inflammatory breast carcinoma), ovarian cancer (e.g., epithelial ovarian cancer, extragonadal germ cell tumor, ovarian germ cell tumor, low-grade ovarian tumor), prostate cancer (e.g., hormone-dependent prostate cancer, hormone-independent prostate cancer), liver cancer (e.g., primary liver cancer, extrahepatic bile duct cancer), thyroid cancer (e.g., medullary thyroid cancer), kidney cancer (e.g., renal cell carcinoma, transitional cell carcinoma of renal pelvis and ureter), uterine cancer, brain tumor (e.g., pineal astrocytoma, pilocytic astrocytoma, diffuse astrocytoma, anaplastic astrocytoma), melanoma, sarcoma, bladder cancer, hematological cancer or the like including multiple myeloma, pituitary adenoma, glioma, acoustic neurinoma, retinal sarcoma, pharyngeal cancer, laryngeal cancer, tongue cancer, thymoma, esophageal carcinoma, duodenal cancer, colon cancer, rectal cancer, hepatocellular carcinoma, pancreatic endocrine tumor, bile duct cancer, gallbladder cancer, penile cancer, ureteric cancer, testicular tumor, vulvar carcinoma, cervix cancer, corpus uteri carcinoma, uterine sarcoma, trophoblastic disease, vaginal cancer, skin cancer, mycosis fungoides, basalioma, soft part sarcoma, malignant lymphoma, Hodgkin's disease, myelodysplastic syndrome, adult T-cell leukemia, chronic myeloproliferative disease, pancreatic endocrine tumor, fibrous histiocytoma, leiomyosarcoma, rhabdomyosarcoma, carcinoma of unknown primary), leukaemia (e.g., acute leukaemia (e.g., acute lymphocytic leukaemia, acute myeloid leukaemia), chronic leukaemia (e.g., chronic lymphocytic leukaemia, chronic myeloid leukaemia), myelodysplastic syndrome), uterine sarcoma (e.g., uterine mesodermal mixed tumor, uterine leiomyosarcoma, endometrial stromal tumor), myelofibrosis, and so on].

### Pharmaceutical Compositions

The composition of the present invention as a drug may be produced by using a method as disclosed herein with a pharmaceutically acceptable carrier. Examples of the dosage form of the drug include formulations for oral administration (e.g., a capsule, tablet, or liquid) blended with a conventional auxiliary such as a buffering agent and/or a stabilizer. Examples of the dosage form of the drug include formulations for parenteral administration (e.g., a liquid such an injection) blended with a conventional auxiliary such as a buffering agent and/or a stabilizer. Examples of the dosage form of the drug include formulations for topical administration, such as an ointment, a cream, a liquid, and a plaster, blended with a conventional pharmaceutical carrier.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable excipient,” refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The composition of the present invention may be used for introduction of an active ingredient into various types of cells, tissues, or organs. Examples of cells to which the composition of the present invention may be applied include mesenchymal stem cells, neural stem cells, skin stem cells, splenocytes, nerve cells, glial cells, pancreatic B cells, bone marrow cells, mesangial cells, Langerhans cells, epidermal cells, epithelial cells, endothelial cells, fibroblasts, fiber cells, muscle cells (e.g., skeletal muscle cells, cardiac muscle cells, myoblasts, muscle satellite cells, smooth muscle cells), fat cells, blood cells (e.g., macrophages, T cells, B cells, natural killer cells, mast cells, leukocytes, neutrophils, basophils, eosinophils, monocytes, megakaryocytes, hematopoietic stem cells), synoviocytes, chondrocytes, osteocytes, osteoblasts, osteoclasts, mammary cells, hepatocytes or stromal cells, ova, spermatids, or precursor cells capable of inducing differentiation into these cells, stem cells (e.g., including induced pluripotent stem cells (iPS cells), embryonic stem cells (ES cells)), primordial germ cells, oocytes, and fertilized ova. Examples of tissues or organs to which the composition of the present invention may be applied include all tissues or organs in which the above cells are present, for example, brain, sites of brain (e.g., olfactory bulb, amygdala, basal ganglion, hippocampus, thalamus, hypothalamus, subthalamic nucleus, cerebral cortex, medulla oblongata, cerebellum, occipital lobe, frontal lobe, temporal lobe, putamen, caudate nucleus, callosum, substantia nigra), spinal cord, pituitary gland, stomach, pancreas, kidney, liver, gonad, thyroid, gallbladder, bone marrow, adrenal gland, skin, lung, digestive tract (e.g., large intestine, small intestine), blood vessel, heart, thymus, spleen, submandibular gland, peripheral blood, peripheral blood cells, prostate, placenta, uterus, bones, joints, and muscles (e.g., skeletal muscle, smooth muscle, cardiac muscle).

The composition of the present invention is particularly superior in efficiency to introduce a nucleic acid into target cells.

The compound, the lipid particle, and the composition of the present invention are stable and have low toxicity and can be safely used. In using the composition of the present invention in vivo, or using the composition as a drug, the composition is suitably administered to a subject (e.g., a human or a non-human mammal (preferably, a human)) so that an effective amount of the nucleic acid can be delivered to targeted cells.

In using the composition of the present invention in vivo, or using the composition as a drug, the composition can be orally or parenterally (e.g., local, rectal, or intravenous administration) administered in a safe manner in the form of a pharmaceutical formulation such as tablets (including sugar-coated tablets, film-coated tablets, sublingual tablets, orally disintegrating tablets), powders, granules, capsules (including soft capsules, microcapsules), liquids, troches, syrups, emulsions, suspensions, injections (e.g., subcutaneous injections, intravenous injections, intramuscular injections, intraperitoneal injections), topical formulations (e.g., formulations for nasal administration, transdermal formulations, ointments), suppositories (e.g., rectal suppositories, vaginal suppositories), pellets, nasal formulations, pulmonary formulations (inhalations), and infusions. Each formulation may be a controlled-release formulation such as a fast-release formulation and a sustained-release formulation (e.g., a sustained-release microcapsule).

An “effective amount” or “therapeutically effective amount” of an active agent or therapeutic agent such as a therapeutic nucleic acid is an amount sufficient to produce the desired effect, e.g. an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as siRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

### Methods of Producing LNP

The composition of the present invention provides methods of producing a pharmaceutical composition, such as lipid nanoparticles, as disclosed herein. In some embodiments, the method of producing a pharmaceutical composition, comprising a. producing lipid nanoparticle (LNP) by mixing a lipid mixture and an aqueous solution, wherein the lipid mixture comprises: i. a cationic ionizable lipid, wherein the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA); ii. a helper lipid, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dimyristoyl-sn-glycero-phosphocholine (DMPC), and 1,2-dipalmitoyl-sn-glycero-3-phosphocholine (DPPC); iii. a sterol, wherein the sterol is selected from the group consisting of cholesterol, β-sitosterol, campesterol, ergosterol, phytosterols (e.g., stigmastanol), and fucosterol; iv. a conjugated lipid, wherein the conjugated lipid is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K, and PEG-DMG-5000K; and the aqueous solution comprises: iv. a nucleic acid, wherein the nucleic acid is DNA and at least 2 kilobases (kb), at least 3 kb, at least 4 kb, at least 5 kb, at least 5 kb, at least 7 kb, at least 8 kb, at least 9 kb, or at least 10 kb; and b. mixing the LNPs produced in step a) and a pharmaceutically acceptable carrier.

In some embodiments, the aqueous solution is a buffer selected from the group consisting of sodium acetate buffer, HEPES (4-(2-hydroxyethyl)-1-piperazineethanesulfonic acid), PBS (phosphate buffered saline), TRIS (tris(hydroxymethyl)aminomethane), MES (2-(N-morpholino) ethanesulfonic acid), and citrate buffer. In one embodiment, the aqueous solution is sodium acetate buffer. In one embodiment, the aqueous solution is PBS.

In some embodiments, the aqueous solution has a pH of about pH 5.0 to about pH 8.0. In some embodiments, the pH is about 5.0.

According to the present invention, a lipid solution contains a mixture of lipids suitable to form lipid nanoparticles for encapsulation of nucleic acid (e.g., DNA or RNA). In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

In some embodiments, the nucleic acid molecule is DNA or RNA. In some embodiments, the nucleic acid molecule is DNA.

In some embodiments, the LNP composition further comprises a sugar (e.g., the LNPs are in an aqueous solution comprising sugar). In some embodiments, the sugar is selected from the group consisting of lactose, sucrose, trehalose, and a combination thereof.

In some embodiments, the amount of sugar in the LNP composition is between about 0% w/v and about 40% w/v. In one embodiment, the amount of sugar is between about 0.1% w/v and about 40% w/v, about 1% w/v to about 40% w/v, about 1% w/v to about 35% w/v, about 1% w/v to about 30% w/v, about 1% w/v to about 25% w/v, about 1% w/v to about 20% w/v, about 1% w/v to about 15% w/v, about 1% w/v to about 10% w/v, about 1% w/v to about 5% w/v, about 5% w/v to about 40% w/v, about 5% w/v to about 35% w/v, about 5% w/v to about 30% w/v, about 5% w/v to about 25% w/v, about 5% w/v to about 20% w/v, about 5% w/v to about 15% w/v, about 5% w/v to about 10% w/v, about 10% w/v to about 40% w/v, about 10% w/v to about 35% w/v, about 10% w/v to about 30% w/v, about 10% w/v to about 25% w/v, about 10% w/v to about 20% w/v, about 10% w/v to about 15% w/v, about 15% w/v to about 40% w/v, about 15% w/v to about 35% w/v, about 15% w/v to about 30% w/v, about 15% w/v to about 25% w/v, about 15% w/v to about 20% w/v, about 20% w/v to about 40% w/v, about 20% w/v to about 35% w/v, about 20% w/v to about 30% w/v, about 20% w/v to about 25% w/v, about 25% w/v to about 40% w/v, about 25% w/v to about 35% w/v, about 25% w/v to about 30% w/v, about 30% w/v to about 40% w/v, about 30% w/v to about 35% w/v, or about 35% w/v to about 40% w/v. In one embodiment, the amount of sugar is at least 5% w/v, at least 10% w/v, at least 20% w/v, or at least 30% w/v. In one embodiment, preferably the amount of sugar is about 5% w/v.

### Lyophilization

In some embodiments, the pharmaceutical composition is lyophilized and reconstituted for administration to a subject. In some embodiments, the lyophilized pharmaceutical composition that, when reconstituted, has a minimal amount of large aggregates. Such large aggregates may have a size greater than about 0.2 μm, greater than about 0.5 μm, or greater than about 1 μm, and can be undesirable in a reconstituted solution. Aggregate sizes can be measured using a variety of techniques including those indicated in the U.S. Pharmacopeia 32<788>, hereby incorporated by reference. The tests may include a light obscuration particle count test, microscopic particle count test, laser diffraction, and single particle optical sensing. In one embodiment, the particle size in a given sample is measured using laser diffraction and/or single particle optical sensing. DLS may be used to measure particle size, but it relies on Brownian motion so the technique may not detect some larger particles. Laser diffraction relies on differences in the index of refraction between the particle and the suspension media. The technique is capable of detecting particles at the sub-micron to millimeter range. Relatively small (e.g., about 1-5 wt %) amounts of larger particles can be determined in nanoparticle suspensions. Single particle optical sensing (SPOS) uses light obscuration of dilute suspensions to count individual particles of about 0.5 pm. By knowing the particle concentration of the measured sample, the weight percentage of aggregates or the aggregate concentration (particles/mL) can be calculated.

Formation of aggregates can occur during the freezing and/or drying steps of lyophilization, e.g., due to the dehydration of the surface of the particles. The freezing process has a concentrating effect that can reduce the distance between the particles as the ice forms (Alison et al., Biochim Biophys Acta. 2000 Sep. 29; 1468 (1-2): 127-38; Armstrong and Anchordoquy, J Pharm Sci. 2004 November; 93 (11): 2698-709). This dehydration can be avoided by using lyoprotectants, such as polysaccharides, in the suspension before lyophilization. Suitable polysaccharides include sucrose, lactulose, lactose, maltose, trehalose, or cellobiose, kojibiose, nigerose, isomaltose, trehalose, sophorose, laminaribiose, gentiobiose, turanose, maltulose, palatinose, gentiobiulose, mannobiase, melibiose, melibiulose, rutinose, rutinulose, and xylobiose. In one embodiment, the composition comprises a polysaccharide that is sucrose. In another embodiment, the composition comprises a polysaccharide that is trehalose. Applicants results show, when compared to the starting suspension, that equivalent DLS size distributions are obtained upon reconstitution.

In another aspect, the invention provides a method of preventing substantial aggregation of particles in a pharmaceutical nanoparticle composition comprising adding a sugar and a salt to the lyophilized formulation to prevent aggregation and release of nucleic acid from the interior of the liposome

The lyophilized pharmaceutical composition comprising LNPs can be reconstituted in an appropriate buffers, for example, but not limited to, water, PBS, and HEPES. In some embodiments, the lyophilized pharmaceutical composition comprising LNPs is reconstituted in buffer at a pH between about 5.0 and 8.0. In some embodiments, the pH is 7.4.

### Nebulization

In some embodiments, a device is used to administer the formulations to the lungs. Suitable devices include, but are not limited to, dry powder inhalers, pressurized metered dose inhalers, nebulizers, and electrohydrodynamic aerosol devices.

The liquid formulations comprising the LNPs described above can also be administered using a nebulizer. Nebulizers are liquid aerosol generators that convert the liquid formulation described able, usually aqueous-based compositions, into mists or clouds of small droplets, preferably having diameters less than 5 microns mass median aerodynamic diameter, which can be inhaled into the lower respiratory tract. This process is called atomization. The droplets carry the one or more active agents into the nose, upper airways or deep lungs when the aerosol cloud is inhaled. Any type of nebulizer may be used to administer the formulation to a patient, including, but not limited to pneumatic (jet) nebulizers and electromechanical nebulizers.

### Method of Treatment

The present invention provides methods of treating a subject by administering the composition as disclosed herein. The lyophilized composition, as disclosed herein, can be administered orally, via nebulization, intravenously, subcutaneously, intramuscularly, intraperitoneally, topically, and by infusions.

The term “patient” refers to a subject who may seek or be in need of treatment, requires treatment, is receiving treatment, will receive treatment, or a subject who is under care by a trained professional for a particular disease or condition.

The terms “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans).

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### Method of Treatment

The present invention provides methods of optimizing a Lipid Nanoparticle (LNP) comprising steps of (1) screening LNPs in vitro and (2) screening LNPs in vivo using immune deficient animal.

The LNP can be optimized from many perspectives including the type of each component, the ratio of the components, the payload encapsulated in the LNP and the N/P ratio.

In some embodiments, LNPs are tested in vitro for transfection efficiency including protein expression followed by an evaluation in the in vivo model using immune deficient animal in the method. Subject LNPs in the in vitro test can be screened to limited number of LNPs and the screened LNPs can be evaluated in the immune deficient animal in vivo model. The parameters to be assessed in the immune deficient animal in vivo model may be selected appropriately by the person skilled in the art. The parameters may include protein expression, payload (e.g., nucleic acid) delivery or distribution, durability of expression, dose titration and toxicity, including immunotoxicity.

In some embodiments, the physicochemical characteristics of LNPs are tested followed by the in vitro testing for transfection efficiency. The physicochemical characteristics may be selected appropriately by the person skilled in the art. The physicochemical characteristics may include the size, charge, N/P ratio and encapsulation efficiency. The parameters may include protein expression and toxicity including immunotoxicity. Subject LNPs can be screened to a reduced number of LNPs through the physicochemical characteristics testing and the screened LNPs can be evaluated in the subsequent in vitro testing or in vivo testing.

In some embodiments, the LNPs are evaluated in an in vivo model using wild type animal following the immune deficient animal in vivo model evaluation. The parameters to be assessed in the wild type animal in vivo model may be selected appropriately by the person skilled in the art. The parameters may include protein expression, payload (e.g., nucleic acid) delivery or distribution, durability of expression, dose titration and toxicity, including immunotoxicity.

In some embodiments, the LNPs are evaluated in an in vivo model using animal having a disease of interest following the wild type animal in vivo model evaluation. The parameters to be assessed in the disease animal in vivo model may be selected appropriately by the person skilled in the art based on the disease of interest. The parameters may include protein expression and any pharmacodynamic parameters that may enable evaluation of the therapeutic effect by the payload in the LNP.

In some embodiments, any part of or whole steps may be repeated to screen LNPs. In some embodiments, the components, ratio of the components, payload of the LNPs may be varied to screen and adjust them.

The animal used in the method is selected appropriately by the person skilled in the art. The animal may be a mouse, rat, rabbit, dog, porcine and/or monkey. As an immune deficient animal, a person skilled in the art can select appropriately from animals with lack of or deficient immune response against the exogenous molecules. The immune deficient animal may be NOD animal, NSG animal, SCID animal, or an animal deficient in specific components of the immune and/or DNA sensing pathways including cGAS knockout animal, STING knockout animal or an animal with knockout elements of the interferon signaling pathway. In some embodiments, NOD mouse, NSG mouse or SCID mouse is preferable.

### EXAMPLES

Additional embodiments of the present disclosure are illustrated in further detail in the following examples, which are not in any way intended to limit the scope of the claims.

### Example 1: LNP (Lipid Nano Particle) Preparation

Ionizable-lipids, helper lipids, cholesterol and PEG-DMG were dissolved in ethanol to make 25 mM stock of each respectively which were than mixed at a molar ratio of 50/10/38.5/1.5. Nucleic acid of interest was diluted in 25 mM sodium acetate buffer (pH 5). The lipid mixture and nucleic acid solution were mixed using a microfluidic micromixer (Precision Nanosystems, Vancouver, BC, Canada) at room temperature with a flow rate ratio of 3 ml/min: 9 mL/min to afford a dispersion containing lipid nanoparticles (LNPs) encapsulating respective nucleic acid. LNPs obtained were either dialyzed or filtered through Amicon U-15 filters (30 kDa or 100 kDa, cellulose) tube at 2000×g 4° C. for 30 min to remove excess ethanol and replace buffer with PBS. Sterile sucrose solution (20% final volume) was added to the formulations as cryoprotectant. Subsequently, formulations were filtered through 0.2 μm syringe filter. Quant-iT™ PicoGreen dsDNA assay kit (Thermo Fisher Scientific) was used as manufacturer's protocol to quantify amount of nucleic acid encapsulated in the LNPs. The particle size and PDI of the LNPs were measured by using a Zetasizer Ultra Zs Xplorer (Malvern Instruments Limited). The formulations were kept at −80° C. until used for in vitro/in vivo experiments.

### Work Flow of Systemic Optimization Approach to Determine LNP Formulations:

In vitro testing of LNP-DNA formulation in hepatic cell line: Hepatic cells were seeded in a 24-well tissue culture plate followed by transfection of 1 μg of DNA contained in corresponding LNP-DNA formulation in a serum free medium. Serum supplemented media was added post transfection and transgene expression and viability was measured after 24 hrs of transfection.

In vivo testing of LNP-DNA formulation: Adult male mice were injected intravenously at 10 ml/kg and monitored for 72 hrs post dosing for clinical observations. Terminal collections at specified time points included serum and perfused liver tissue for analysis of transgene expression and DNA content.

Table 1 shows in vitro screening stages for LNP-DNA vectors optimization.

TABLE 1

| Amino Lipids | Helper Lipids | N:P ratio | Payload npDNA |
|---|---|---|---|
| MC3 | DSPC | 4, 6, 8, 10 | CAG-Luc |
| KC2 | | | LSP₁-hATP7B(wt) |
| Lipid 1 | | | |
| MC3 | DSPC | 4, 5, 6 | LSP₁-hATP7b(wt) |
| | DOPC | | |
| | DOPE | | |
| | DMPC | | |
| | DDPC | | |
| MC3 | DOPC | 4 | LSP₁-hATP7b(wt) |
| KC2 | | | LSP₁-hATP7b(CodOpt) |
| Lipid 1 | | | LSP₁-hATP7b(Trunc) |
| | | | LSP₂-eGFP(wt) |

N/P ratio of LNP formulations including amino lipids and DSPC were optimal at approximately 4-6 and resulted in mild expression. Lipid 1-DSPC resulted with severe toxicity. MC3-DOPC and MC3-DOPE resulted in surprising increased protein expression at a N/P ratio of 4. Moderate toxicity was seen with MC3-DSPC formulation.

Lipid 1-DOPC consistently produced high levels of hATP7B and eGFP. Codon optimized hATP7B was also determined to result in surprisingly higher expression than wild type sequence.

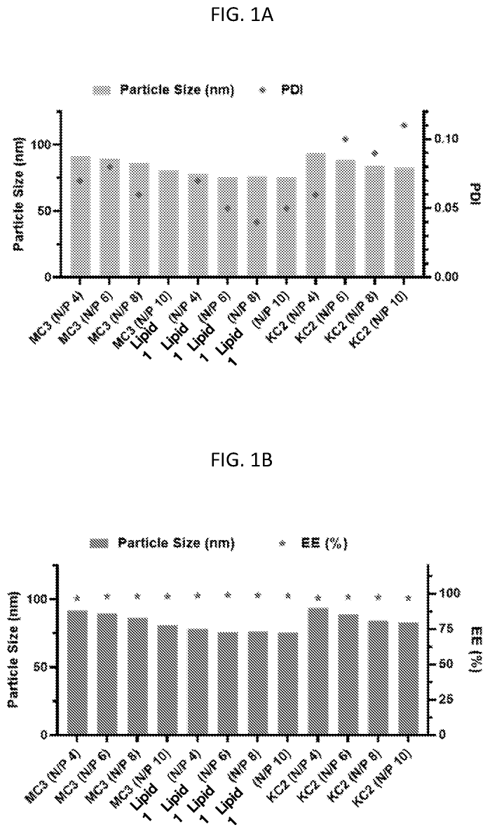

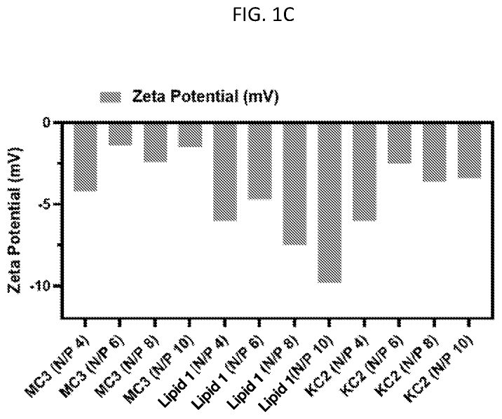

Table 2 shows LNP formulations and physical properties as shown in FIGS. 1A-1C. Ionizable lipids, helper lipids, sterol, and PEG2K-DMG were at a lipid molar ratio of 50%: 10%: 38.5%: 1.5%.

TABLE 2

| | Lipids | | | | | | | | Zeta |
|---|---|---|---|---|---|---|---|---|---|
| | | | | PEG | N/P | Size | | EE | Potential |
| Cargo | Ionizable | Helper | Sterol | lipid | Ratio | (nm) | PDI | (%) | (mV) |
| CAG- | MC3 | DSPC | Chol | PEG2K- | 4 | 76.5 | 0.05 | 84.3 | −6.4 |
| Luc | | | | DMG | | (79.2) | (0.04) | | |
| CAG- | MC3 | DSPC | Chol | PEG2K- | 6 | 64.7 | 0.06 | 97 | −2.7 |
| Luc | | | | DMG | | (73.7) | (0.04) | | |
| CAG- | MC3 | DSPC | Chol | PEG2K- | 8 | 65.6 | 0.04 | 96.5 | −1.7 |
| Luc | | | | DMG | | (70.6) | (0.02) | | |
| CAG- | MC3 | DSPC | Chol | PEG2K- | 10 | 58.9 | 0.08 | 97.3 | −0.4 |
| Luc | | | | DMG | | (63.5) | (0.07) | | |
| ATP7B | MC3 | DSPC | Chol | PEG2K- | 4 | 91.7 | 0.07 | 96.7 | −4.2 |
| | | | | DMG | | | | | |
| ATP7B | MC3 | DSPC | Chol | PEG2K- | 6 | 89.7 | 0.08 | 98.3 | −1.4 |
| | | | | DMG | | | | | |
| ATP7B | MC3 | DSPC | Chol | PEG2K- | 8 | 86.5 | 0.06 | 98.2 | −2.4 |
| | | | | DMG | | | | | |
| ATP7B | MC3 | DSPC | Chol | PEG2K- | 10 | 80.9 | 0.14 | 98.1 | −1.5 |
| | | | | DMG | | | | | |
| ATP7B | Lipid1 | DSPC | Chol | PEG2K- | 4 | 78.3 | 0.07 | 98.5 | −6.0 |
| | | | | DMG | | | | | |
| ATP7B | Lipid1 | DSPC | Chol | PEG2K- | 6 | 75.7 | 0.05 | 98.9 | −4.7 |
| | | | | DMG | | | | | |
| ATP7B | Lipid1 | DSPC | Chol | PEG2K- | 8 | 76.3 | 0.04 | 98.9 | −7.5 |
| | | | | DMG | | | | | |
| ATP7B | Lipid1 | DSPC | Chol | PEG2K- | 10 | 75.5 | 0.05 | 98.5 | −9.8 |
| | | | | DMG | | | | | |
| ATP7B | KC2 | DSPC | Chol | PEG2K- | 4 | 93.7 | 0.06 | 96.9 | −6.0 |
| | | | | DMG | | | | | |
| ATP7B | KC2 | DSPC | Chol | PEG2K- | 6 | 88.8 | 0.10 | 97.7 | −2.5 |
| | | | | DMG | | | | | |
| ATP7B | KC2 | DSPC | Chol | PEG2K- | 8 | 84.3 | 0.09 | 97.4 | −3.6 |
| | | | | DMG | | | | | |
| ATP7B | KC2 | DSPC | Chol | PEG2K- | 10 | 83.1 | 0.11 | 96.9 | −3.4 |
| | | | | DMG | | | | | |

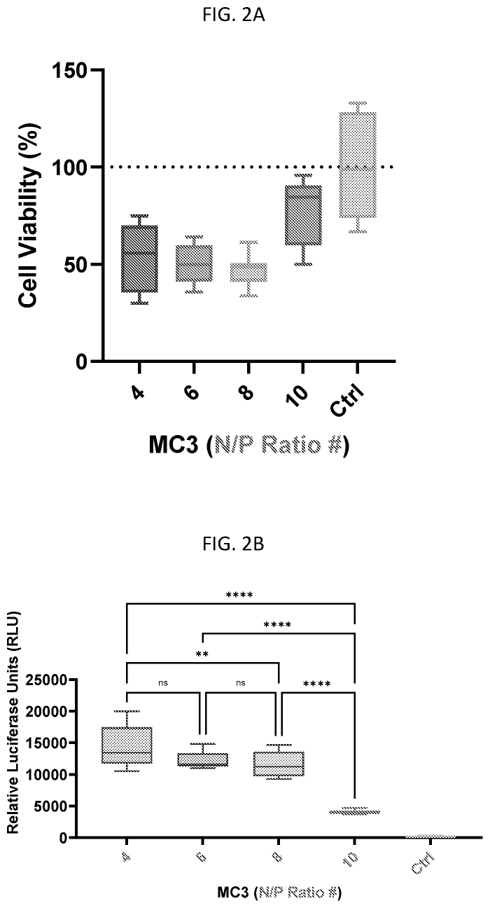

Impact of LNP-npDNA Formulations at Different N/P Ratio on Transgene Expression in Huh7 Cells. FIG. 2A shows percentage of viable cells as function of N/P ratio following incubation with LNP-npDNA (CAG-Luc) containing MC3 as ionizable-lipid. FIG. 2B shows luciferase expression as function of N/P ratio following incubation with LNP-npDNA (CAG-Luc) containing MC3 as ionizable-lipid.

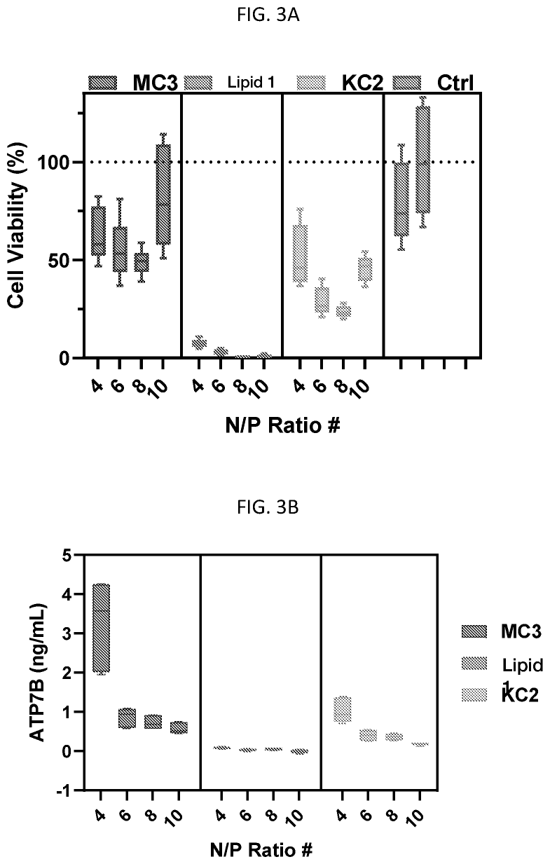

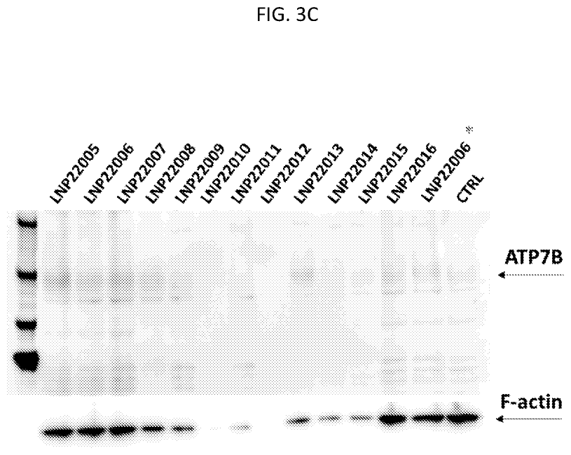

Impact of LNP-npDNA Formulations Containing Varying Ionizable Lipids at Different N/P Ratio on Transgene Expression in Huh7 Cells. FIG. 3A shows the percentage of viable cells as function of N/P ratio following incubation with LNP-npDNA (ATP7B) containing ionizable-lipids shown in legend. FIGS. 3B and 3C DNA expression as function of N/P ratio following incubation with LNP-npDNA (ATP7B) containing ionizable-lipids shown in legend. Treatment of Huh7 cells were performed using 50 μg/mL stock instead of 25 μg/mL stock for other groups.

Table 3 shows LNP formulations and physical properties screening helper lipids. Ionizable lipids, helper lipids, sterol, and PEG2K-DMG were at a lipid molar ratio of 50%: 10%: 38.5%: 1.5%.

TABLE 3

| | Lipids | | | | N/P | Size | | EE |
|---|---|---|---|---|---|---|---|---|
| Cargo | Ionizable | Helper | Sterol | PEG lipid | Ratio | (nm) | PDI | (%) |
| CAG-Luc | Lipid 1 | DSPC | Chol | PEG2K-DMG | 6 | 87.43 | 0.138 | 95.4 |
| ATP7B | MC3 | DSPC | Chol | PEG2K-DMG | 4 | 94.85 | 0.065 | 92.6 |
| ATP7B | MC3 | DSPC | Chol | PEG2K-DMG | 5 | 85.09 | 0.069 | 92.6 |
| ATP7B | MC3 | DSPC | Chol | PEG2K-DMG | 6 | 81.39 | 0.099 | 94.4 |
| ATP7B | MC3 | DOPC | Chol | PEG2K-DMG | 4 | 105.9 | 0.059 | 93.6 |
| ATP7B | MC3 | DOPC | Chol | PEG2K-DMG | 5 | 104.9 | 0.026 | 96.3 |
| ATP7B | MC3 | DOPC | Chol | PEG2K-DMG | 6 | 88.47 | 0.071 | 95.4 |
| ATP7B | MC3 | DOPE | Chol | PEG2K-DMG | 4 | 114.8 | 0.028 | 94.4 |
| ATP7B | MC3 | DOPE | Chol | PEG2K-DMG | 5 | 108.5 | 0.056 | 95.5 |
| ATP7B | MC3 | DOPE | Chol | PEG2K-DMG | 6 | 109.5 | 0.033 | 95.9 |
| ATP7B | MC3 | DMPC | Chol | PEG2K-DMG | 4 | 91.91 | 0.064 | 89.3 |
| ATP7B | MC3 | DMPC | Chol | PEG2K-DMG | 5 | 86.58 | 0.074 | 91 |
| ATP7B | MC3 | DMPC | Chol | PEG2K-DMG | 6 | 92.43 | 0.076 | 91.3 |
| ATP7B | MC3 | DPPC | Chol | PEG2K-DMG | 4 | 92.54 | 0.069 | 88.8 |
| ATP7B | MC3 | DPPC | Chol | PEG2K-DMG | 5 | 88.88 | 0.079 | 90.4 |
| ATP7B | MC3 | DPPC | Chol | PEG2K-DMG | 6 | 84.44 | 0.084 | 92.1 |

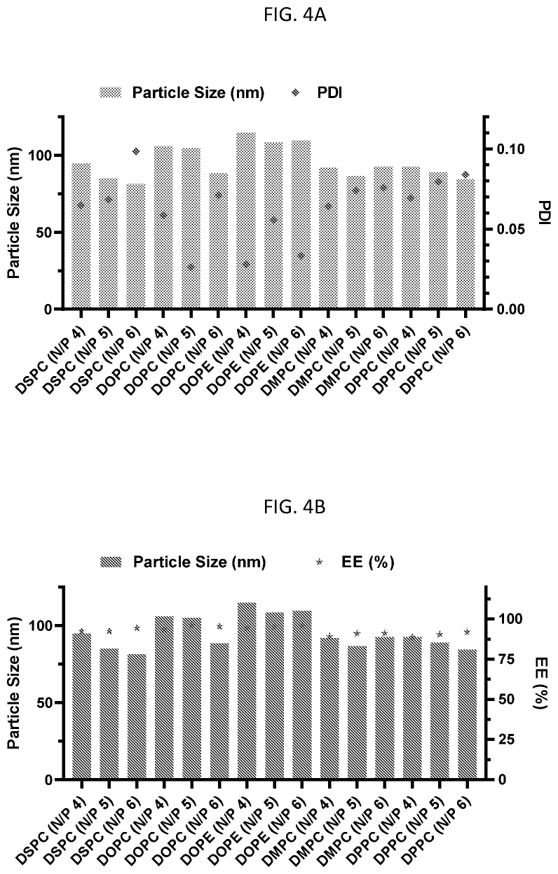

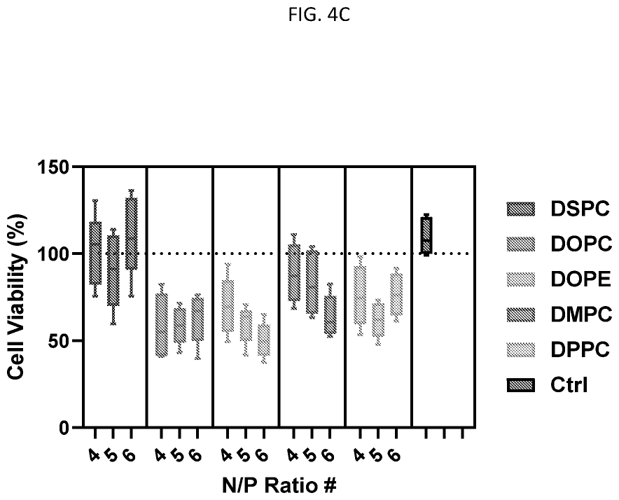

Physio-Chemical Properties and Cell Viability of LNP-npDNA (ATP7B) Systems Containing MC3 and Different Helper Lipids in Huh7 Cells. FIG. 4A shows LNP-npDNA (ATP7B) physio-chemical properties, including particle size and PDI of LNP formulations. FIG. 4B shows dependence of particle size (bar) and encapsulation efficiency (star symbol) on N/P ratio and helper lipids for LNP formulations. FIG. 4C shows cell viability as function of on N/P ratio and helper lipids for LNP formulations.

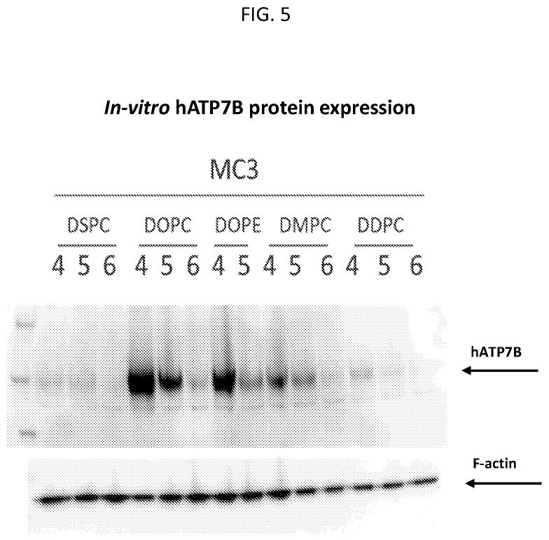

Table 4 shows helper lipid screening as shown in FIG. 5.

TABLE 4

| Amino Lipids | Helper Lipids | N:P ratio | Payload npDNA |
|---|---|---|---|
| MC3 | DSPC | 4, 5, 6 | LSP₁-hATP7b(wt) |
| | DOPC | | |
| | DOPE | | |
| | DMPC | | |
| | DDPC | | |

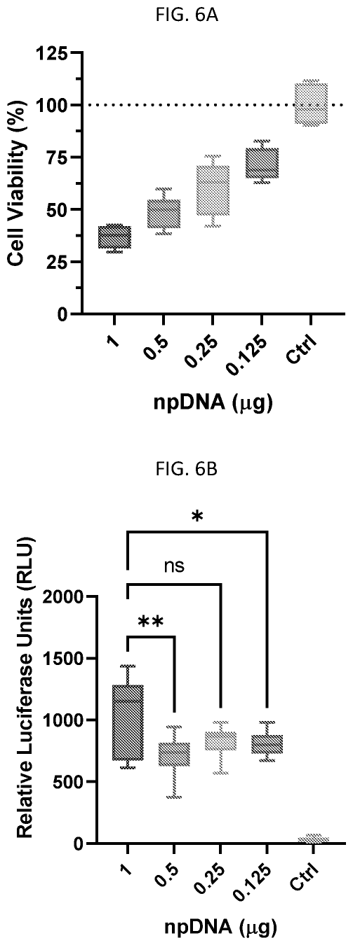

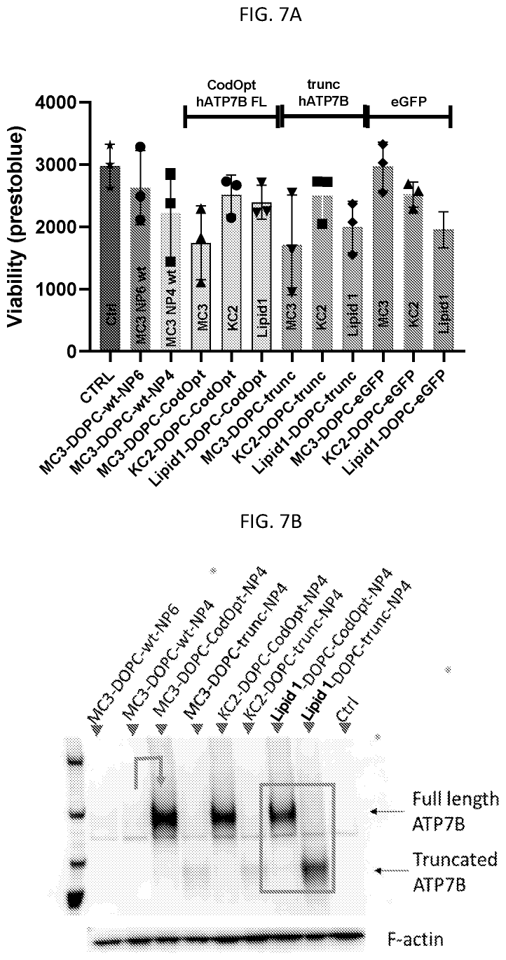

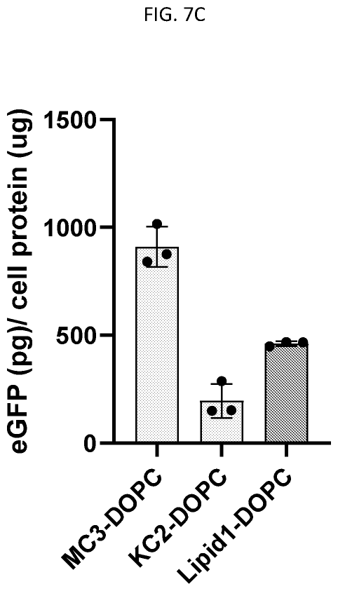

LNP-npDNA (CAG-Luc) System Containing Lipid 1 Ionizable Lipid-DSPC Exhibit Cell Viability and Transfection Dose Dependence in Huh7 Cells. FIG. 6A shows cell viability dose response for Lipid 1 Ionizable Lipid-DSPC. FIG. 6B shows the impact of LNP-npDNA (CAG-Luc) dose on transgene expression.

Table 5 shows amino lipid comparison with optimal helper lipid and N/P ratio. Ionizable lipids, helper lipids, sterol, and PEG2K-DMG were at a lipid molar ratio of 50%: 10%: 38.5%: 1.5%.

TABLE 5

| | Lipids | | | | N/P | Size | | EE |
|---|---|---|---|---|---|---|---|---|
| Cargo | Ionizable | Helper | Sterol | PEG lipid | Ratio | (nm) | PDI | (%) |
| ATP7B_WT | MC3 | DOPC | Chol | PEG2K- | 6 | 105.4 | 0.15 | 97.0 |
| | | | | DMG | | | | |
| ATP7B_WT | MC3 | DOPC | Chol | PEG2K- | 4 | 136 | 0.16 | 94.3 |
| | | | | DMG | | | | |
| ATP7B | MC3 | DOPC | Chol | PEG2K- | 4 | 126.1 | 0.11 | 92.8 |
| (CO^{SEQ4}^{_{—}}^{0CpG}) | | | | DMG | | | | |
| hATP7B_{Truncated} | MC3 | DOPC | Chol | PEG2K- | 4 | 102.1 | 0.05 | 97.5 |
| | | | | DMG | | | | |
| EGFP | MC3 | DOPC | Chol | PEG2K- | 4 | 119.5 | 0.10 | 96.4 |
| | | | | DMG | | | | |
| ATP7B | KC2 | DOPC | Chol | PEG2K- | 4 | 143.1 | 0.18 | 93.5 |
| (CO^{SEQ4}^{_{—}}^{0CpG}) | | | | DMG | | | | |
| hATP7B_{Truncated} | KC2 | DOPC | Chol | PEG2K- | 4 | 103.4 | 0.11 | 96.2 |
| | | | | DMG | | | | |
| EGFP | KC2 | DOPC | Chol | PEG2K- | 4 | 137.7 | 0.22 | 96.8 |
| | | | | DMG | | | | |
| ATP7B | Lipid 1 | DOPC | Chol | PEG2K- | 4 | 112.3 | 0.18 | 97.5 |
| (CO^{SEQ4}^{_{—}}^{0CpG}) | | | | DMG | | | | |
| hATP7B_{Truncated} | Lipid 1 | DOPC | Chol | PEG2K- | 4 | 101.2 | 0.11 | 97.6 |
| | | | | DMG | | | | |
| EGFP | Lipid 1 | DOPC | Chol | PEG2K- | 4 | 109.7 | 0.16 | 97.7 |
| | | | | DMG | | | | |

Table 6 shows amino lipid comparison with optimal helper lipid and N/P combination.

TABLE 6

| Amino Lipids | Helper Lipids | N:P ratio | Payload npDNA |
|---|---|---|---|
| MC3 | DOPC | 4 | LSP1-hATP7b(wt) |
| KC2 | | | LSP1-hATP7b(CodOpt) |
| Lipid 1 | | | LSP1-hATP7b(Trunc) |
| | | | LSP2-eGFP(wt) |

In Vivo Evaluation of DOPC Containing LNP-DNA Formulations with ATP7B and eGFP Payload Nanoplasmids.

Methods: wild type C57 mice were dosed intravenously at indicated dose levels at volume of 10 ml per kg. Serum was collected at 24 hr post dose and body weights and clinical signs were noted as per study protocol. Terminal liver collections were performed at Day 7 post dose and ELISA based protein measurements were used to quantify eGFP and hATP7B expression.

Results: Dose dependent eGFP expression was achieved only with the Lipid1-DOPC-eGFP npDNA formulation as measured by ELISA for eGFP protein is liver tissue from injected mice (0.045 μg/μg liver at 0.3 mg/kg dose to 0.67 pg/μg liver at 1 mg/kg dose).

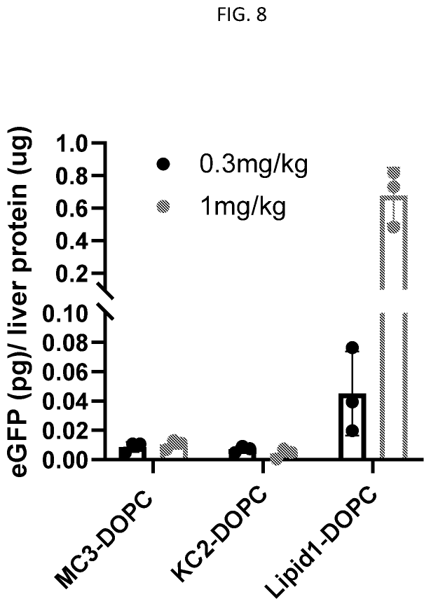

No detectable hATP7B protein was measured in liver samples. (FIG. 8).

LNP-DNA vectors with DOPC helper lipid component were generally well tolerated even at 1 mg/kg.

In Vivo Evaluation of DOPC Containing LNP-DNA Formulations with ATP7B and eGFP Payload Nanoplasmids in Severely Immune Deficient Mice.

Methods: Immune deficient mice purchased from Jackson Laboratories were dosed intravenously at indicated dose levels at volume of 10 ml per kg. Terminal liver collections were performed at Day 7 post dose. Western blotting and ELISA based protein measurements were used to quantify eGFP and hATP7B expression. mRNA and DNA quantification was performed using ddPCR to obtain copy numbers/μg nucleic acid or genomic DNA respectively.

Results: Combined higher dose level (2 mg/kg) was well tolerated by mice with body weight loss well within acceptable limits.

Lipid 1-DOPC formulation was the only successful formulation in achieving in-vivo protein expression of hATP7B which was however 4 fold lower than that measured in human liver. Corresponding mRNA and DNA levels measured in this group was significantly higher compared to nearly background levels measured in MC3 and KC2 containing formulations.

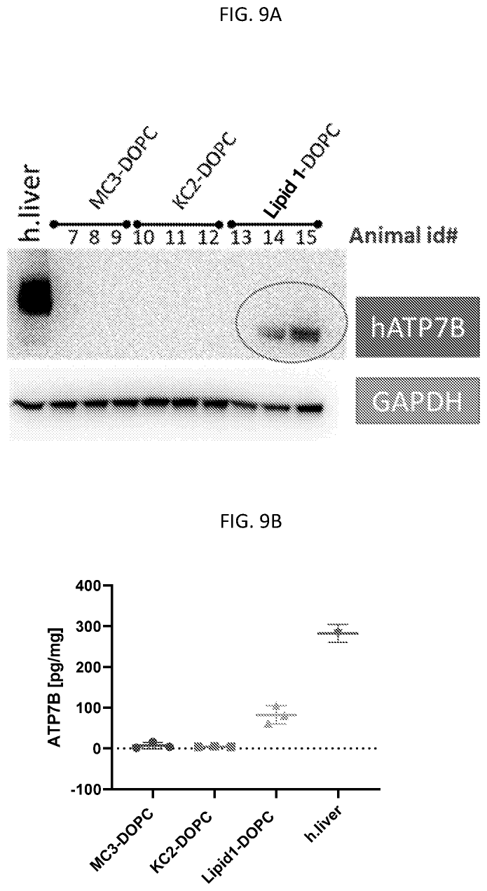

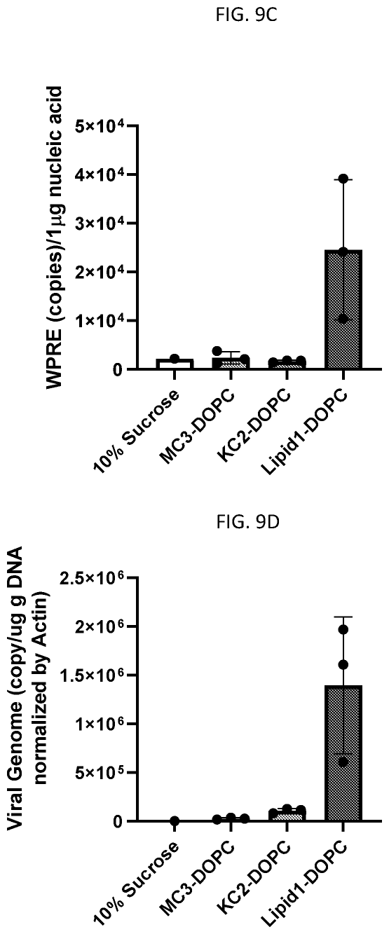

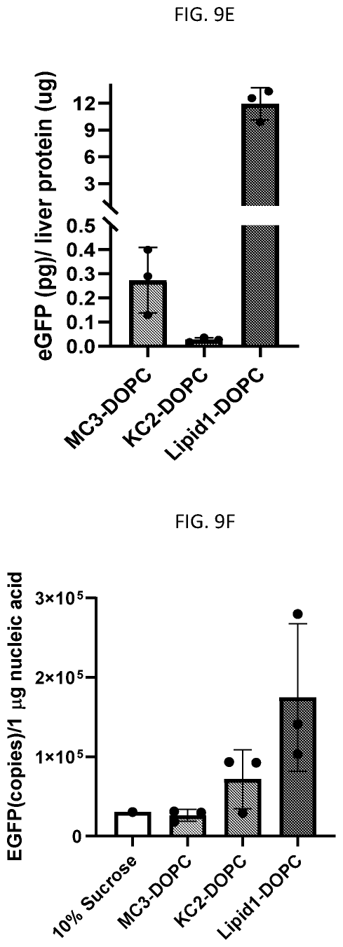

In parallel, the co-dosed reporter formulation of Lipid 1-DOPC with eGFP nanoplasmid payload also outperformed the MC3 and KC2 achieving about 40 fold higher expression than a MC3 lipid formulation. (FIGS. 9A-9F).

Table 7 shows comparative evaluation of multiple transgene expression in vivo utilizing LNP-DNA formulations. Ionizable lipids, helper lipids, sterol, and PEG2K-DMG were at a lipid molar ratio of 50%: 10%: 38.5%: 1.5%.

TABLE 7

| | Lipids | | | | | | | | DNA |
|---|---|---|---|---|---|---|---|---|---|
| | | | | PEG | N/P | Size | | EE | Loading |
| Cargo | Ionizable | Helper | Sterol | lipid | Ratio | (nm) | PDI | (%) | (μg/mL) |
| TTR- | Lipid 1 | DOPC | Chol | PEG2K- | 4 | 108.9 | 0.08 | 98.6 | 643.7 |
| ATP7B | | | | DMG | | | | | |
| ApoE- | Lipid 1 | DOPC | Chol | PEG2K- | 4 | 104.9 | 0.16 | 98.3 | 546.0 |
| AAT- | | | | DMG | | | | | |
| eGFP | | | | | | | | | |
| TTR- | Lipid 1 | DOPC | Chol | PEG2K- | 4 | 94.8 | 0.07 | 97.8 | 406.7 |
| PAH | | | | DMG | | | | | |
| CB- | Lipid 1 | DOPC | Chol | PEG2K- | 4 | 100.4 | 0.06 | 98.2 | 414.1 |
| PAH | | | | DMG | | | | | |
| TTR- | Lipid 1 | DOPC | Chol | PEG2K- | 4 | 96.06 | 0.10 | 98.4 | 415.8 |
| OTC | | | | DMG | | | | | |
| TTR- | Lipid 1 | DOPC | Chol | PEG2K- | 4 | 110.9 | 0.11 | 97.9 | 428.0 |
| OTC- | | | | DMG | | | | | |
| GLA | | | | | | | | | |
| CAG- | Lipid 1 | DOPC | Chol | PEG2K- | 4 | 115.6 | 0.12 | 98.6 | 454.1 |
| FVIII- | | | | DMG | | | | | |
| BDD | | | | | | | | | |
| ApoE- | Lipid 1 | DOPC | Chol | PEG2K- | 4 | 114.9 | 0.10 | 97.5 | 189.4 |
| AAT- | | | | DMG | | | | | |
| FVIII_{—} | | | | | | | | | |
| BDD | | | | | | | | | |

The DNA constructs used include:
HemA constructs:
npZL064 (CAG: FVIII-BDD-SQ: WPRE: polyA: ID_Tag: DTS-SV40)=7,961 bp
npZL065 (ApoE-hAAT: FVIII-BDD-SQ: WPRE: polyA: ID_tag: DTS-SV40)=6,813 bp
PKU construct is with CAG promoter:
npMY166 (CAG: PAH-PK03: WPRE: polyA: ID_tag: DTS-SV40)=4,925 bp

Table 8 shows TA dosing in immune deficient mice (6-8 weeks old male mice).

TABLE 8

| | | | In-life | |
|---|---|---|---|---|
| Test Article/ | Dose | Termi- | serum | Terminal |
| Dose at Day 0 | (mg/kg) | nation | collections | Collections |
| Saline | NA | Day 7 | NA | Day 7 or Day 28 |
| | | Day 28 | Day 7, | collection |
| | | | Day 21 | Serum: Terminal |
| Lipid 1-DOPC | 1.6 + | 4 hr | NA | (maximum) Liver: |
| TTR-ATP7B + | 0.5 | Day 7 | NA | Terminal weight |
| ApoE-AAT-eGFP | | | | Terminal: collected |
| Lipid 1-DOPC | 1.4 | Day 7 | NA | 3 ways Frozen, |
| TTR-PAH | | | | RNALater, 10% |
| Lipid 1-DOPC | 1.4 | Day 7 | NA | formalin fixed |
| CB-PAH | | | | Body weight weekly |
| Lipid 1-DOPC | 1.4 | Day 7 | NA | |
| TTR-OTC | | | | |
| Lipid 1-DOPC | 1.4 | Day 28 | Day 7, | |
| TTR-OTC-GLA | | | Day 21 | |
| Lipid 1-DOPC | 1.4 | Day 28 | Day 7, | |
| CAG-FVIII-BDD | | | Day 21 | |
| Lipid 1-DOPC | 1.4 | Day 28 | Day 7, | |
| ApoE-AAT-FVIII- | | | Day 21 | |
| BDD | | | | |

In-Vivo Evaluation in Immune Deficient Mice of DOPC Containing LNP-DNA Formulations with Multiple Nanoplasmid Constructs of Varying Transgene Sizes and Different Promoter Sequences.

Methods: mice purchased from Jackson Laboratories were dosed intravenously at indicated dose levels at volume of 10 ml per kg. Terminal liver collections were performed at Day 7 post dose for liver resident proteins and Day 28 for secreted proteins. Western blotting, ELISA and immunohistochemistry based protein measurements were used to quantify transgene product expression. mRNA and DNA quantification was performed using ddPCR to obtain copy numbers/μg nucleic acid or genomic DNA respectively. FVIII and GLA protein activity was determined using pre-established assays. Protein measurement for hATP7B, eGFP and hOTC was performed by specific ELISA assays and substantial protein was detected in mouse livers compared to saline treated controls.

Results: *Dosing was mass matched and not copy number matched to account for sequence length differences.

Substantial amounts of npDNA was found resident in liver tissue at Day 7 (~1e6 copies/μg genomic DNA) while lower levels (~1e4 copies/μg gDNA) was observed in the Day 28 samples.

mRNA expressed was positively detected by ddPCR between (1e3 and 1e5 copies/μg nucleic acid) for the different payload constructs as seen in the figure and has a correlation to the size, sequence, promoter used and elapsed time post dose. Larger sized transgenes like ATP7B and FVIII have lesser copies than smaller transgenes like PAH and OTC. For FVIII constructs, the ApoE-AAT promoter had much higher expression compared to CAG promoter. Similarly, Day 28 tissues on average have ~10 fold reduction in mRNA copies although direct comparison of time points was not performed for the same payload.

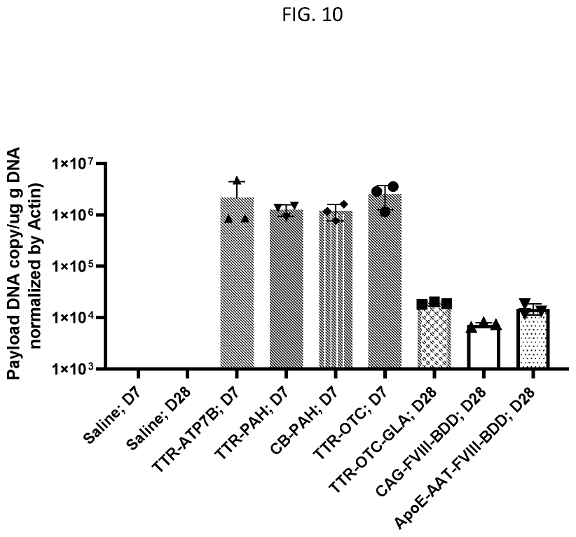

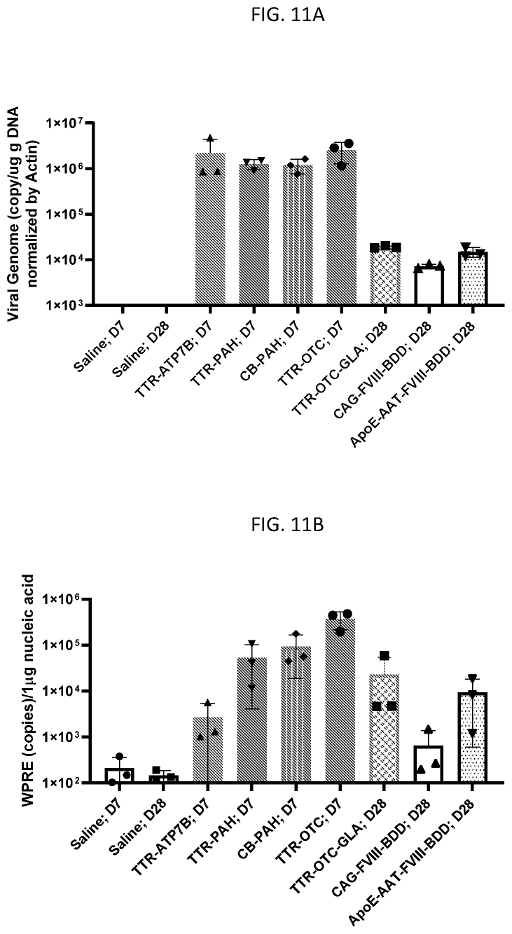

Substantial protein expression was detected in all payloads corresponding to the presence of payload DNA and mRNA expressed. Presence of liver resident proteins OTC, PAH and eGFP was visible in hepatocytes of liver sections as seen by IHC. Activity was secreted proteins FVIII and GLA was detected in serum from Day 7 through Day 28: FVIII activity decreased from 20% at Day 7 to undetected by Day 28 in the CAG promoter construct versus was more stable between 20%-30% for the ApoE-AAT promoter in the same duration. GLA activity on the other hand increased from 100 nM/hr/mL to 300 nM/hr/mL going from Day 7 to Day 28 owing to its better serum stability. Levels of protein expression for hATP7B varied substantially from 36%-128% of human liver levels of hATP7B. (FIGS. 10-11B).

Table 9 shows promoter and transgene constructs.

TABLE 9

| | | Transgene | Construct | | Dose |
|---|---|---|---|---|---|
| Promoter | Trans- | size | size | Dose | (copies/ |
| # | gene# | (bp) | (bp) | (mg/kg) | kg) |
| TTR | hATP7B | 4398 | 6605 | 1.6 | 2.36 E14 |
| ApoE-AAT | eGFP | 720 | 2278 | 0.5 | 2.14 E14 |
| TTR | PAH | 1359 | 3584 | 1.4 | 3.80 E14 |
| CB | PAH | 1359 | 4925 | 1.4 | 2.76 E14 |
| TTR | hOTC | 1065 | 3249 | 1.4 | 4.20 E14 |
| TTR | hGLA hOTC | 2418 | 4605 | 1.4 | 3.00 E14 |
| CAG | FVIII-BDD | 4395 | 7961 | 1.4 | 1.71 E14 |
| ApoE-AAT | FVIII-BDD | 4395 | 6813 | 1.4 | 2.01 E14 |

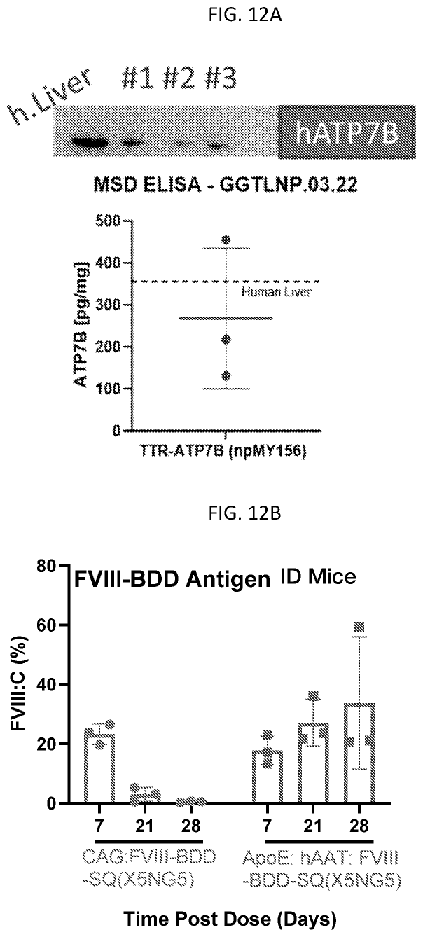

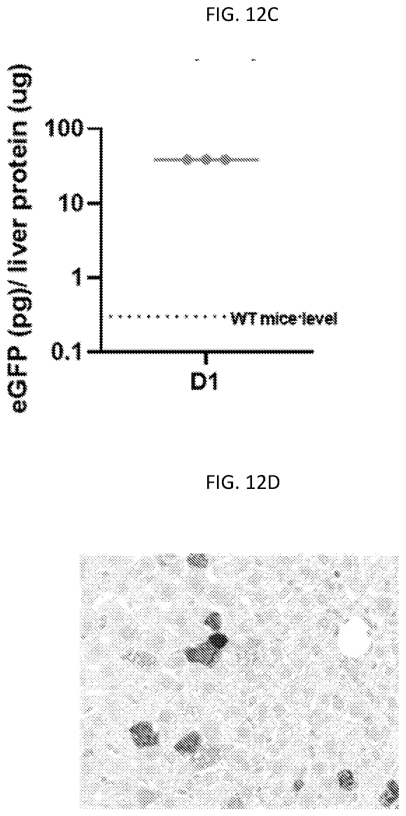

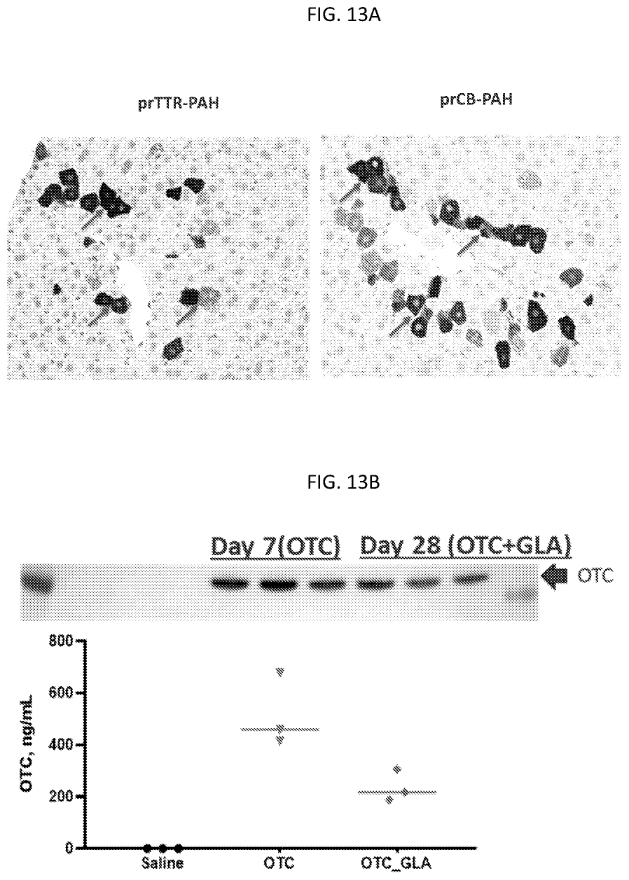

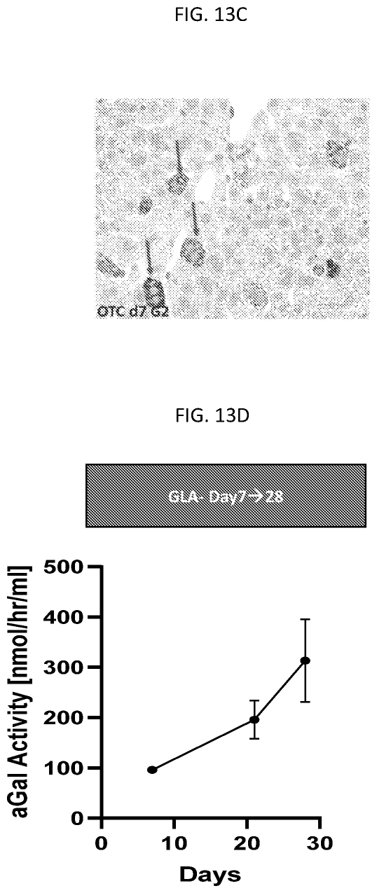

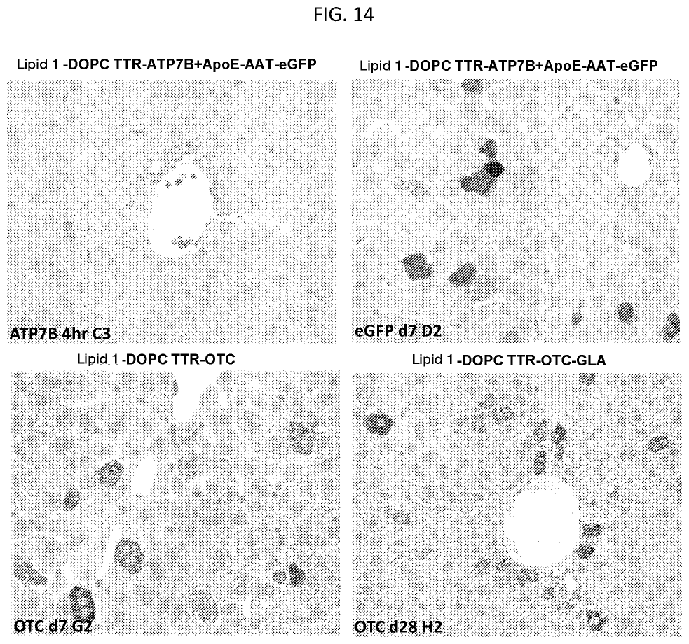

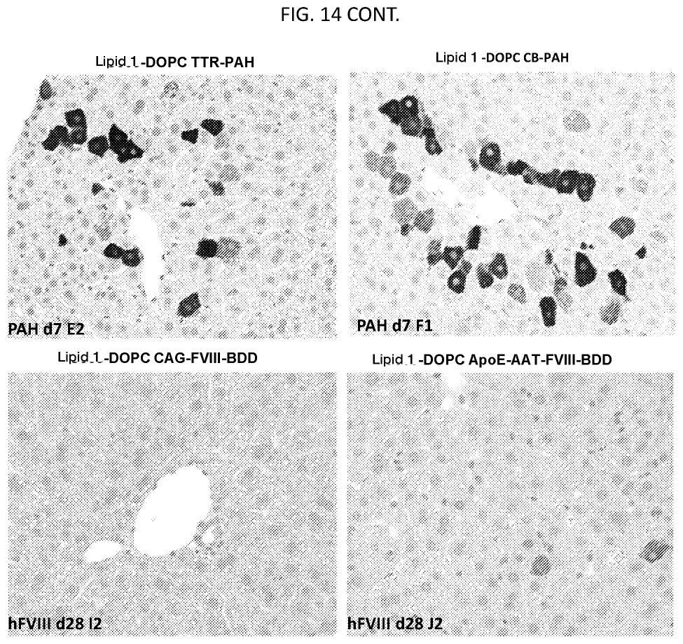

Positive protein readouts were achieved with multiple secreted or liver resident transgenes of varying sizes upon dosing Lipid1-DOPC-npDNA in mice. (FIGS. 12A-15D).

In-vivo evaluation of DOPC containing LNP-DNA formulations with ATP7B, GLA and FVIII payload nanoplasmids in immune-competent WT C57L6 mice.

Methods: wild type C57 mice were dosed intravenously at indicated dose levels at volume of 10 ml per kg. Serum was collected at 24 hr post dose and body weights and clinical signs were noted as per study protocol. Protein measurement was performed by ELISA for hATP7B while GLA and FVIII specific activity assays were performed on collected serum from dosed mice at Day 7.

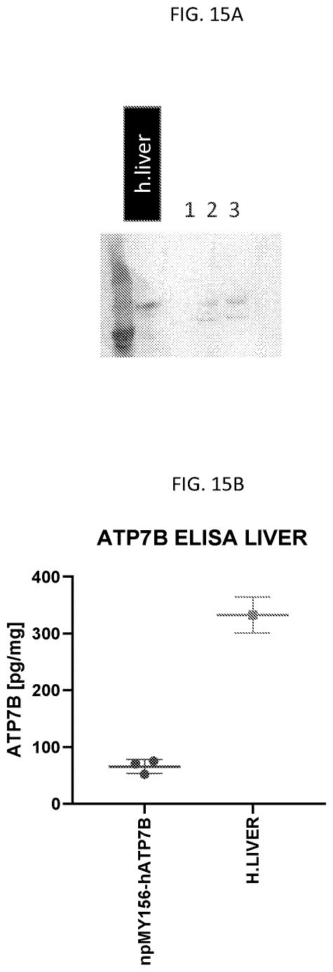

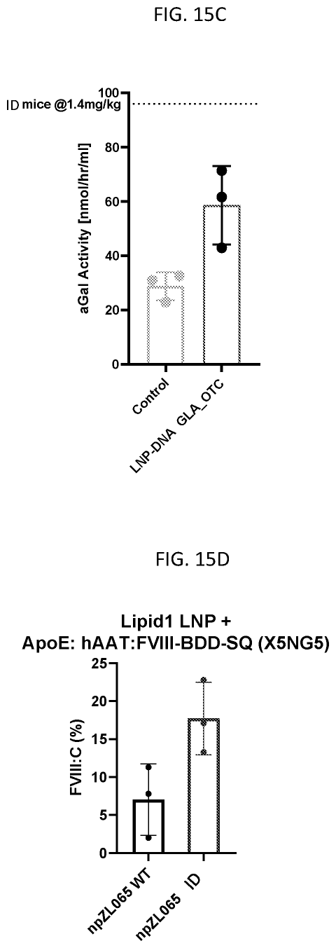

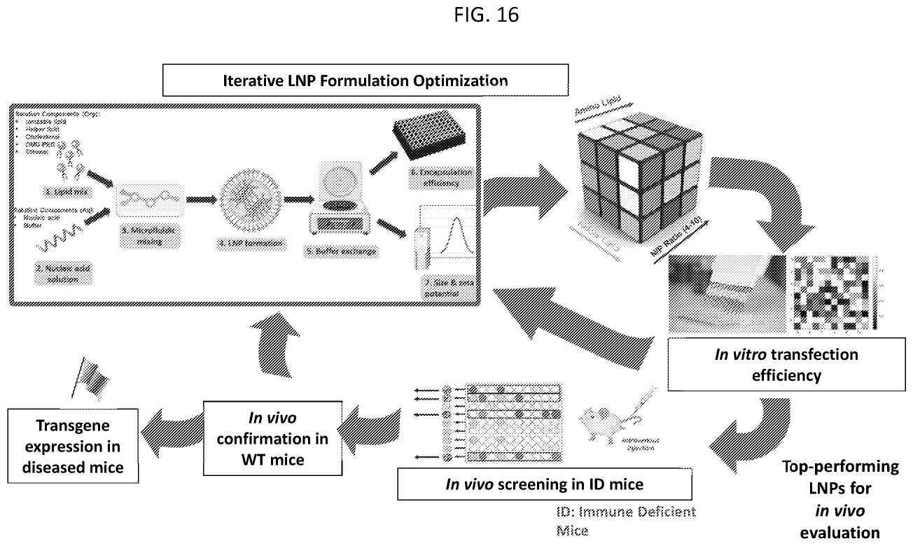

Results: Low detectable levels of hATP7B was observed by western blotting and ELISA measured hATP7B in liver was 5-fold lower than human liver after dose of 1.3 mg/kg. (FIG. 15B)

GLA activity measured was 60 nM/hr/mL of serum collected at Day 7 in wt mice treated with saline (“control”) and with LNP-DNA GLA-OTC (dose: 1.6 mg/kg). GLA activity was 2-3 fold lower than levels observed in immune deficient mice. (FIG. 15C)

hFVIII activity similarly trended lower in WT mice (dose: 2 mg/kg) compared to immune deficient mice (dose: 1.4 mg/kg) and was only measured around 2-11% of normal human level. (FIG. 15D)

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

We claim:

1. A lipid nanoparticle (LNP) composition comprising:
a. a cationic ionizable lipid;
b. a helper lipid, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dimyristoyl-sn-glycero-phosphocholine (DMPC), and 1,2-dipalmitoyl-sn-glycero-3-phosphocholine (DPPC);
c. a sterol, wherein the sterol is selected from the group consisting of cholesterol, β-sitosterol, campesterol, ergosterol, phytosterols, (e.g., stigmastanol), and fucosterol;
d. a conjugated lipid, wherein the conjugated lipid is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K and PEG-DMG-5000K; and
e. a nucleic acid, wherein the nucleic acid is DNA and at least about 2 kilobases (kb), at least about 3 kb, at least about 4 kb, at least about 5 kb, at least about 6 kb, at least about 7 kb, at least about 8 kb, at least about 9 kb, or at least about 10 kb.

2. The LNP composition of claim 1, wherein the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA).

3. The LNP composition of claim 1 or 2, wherein the cationic ionizable lipid is DLin-MC3-DMA.

4. The LNP composition of claim 1 or 2, wherein the cationic ionizable lipid is DLin-K2C-DMA.

5. The LNP composition of any one of claims 1-4, wherein the helper lipid is DOPC.

6. The LNP composition of any one of claims 1-5, wherein the sterol is cholesterol.

7. The LNP composition of any one of claims 1-6, wherein the molar ratio of the cationic ionizable lipid, the helper lipid, the sterol, and the conjugated lipid is about 20%-80%: 5%-20%: 36%-39%: 1%-4% (mol) of the total lipid present in the LNP composition.

8. The LNP composition of any one of claims 1-7, wherein the molar ratio of the cationic ionizable lipid, the helper lipid, the sterol, and the conjugated lipid is about 50%: 10%: 38.5%: 1.5% (mol) of the total lipid present in the LNP composition.

9. The LNP composition of claim 1, wherein the cationic ionizable lipid is DLin-MC3-DMA, the helper lipid is DOPC, the sterol is cholesterol, and the conjugated lipid is PEG-DMG, and wherein the molar ratio of the cationic ionizable lipid, the helper lipid, the sterol, and the PEG-lipid is about 50%: 10%: 38.5%: 1.5% (mol) of the total lipid present in the LNP composition.

10. The LNP composition of claim 1, wherein the cationic ionizable lipid is DLin-KC2-DMA, the helper lipid is DOPC, the sterol is cholesterol, and the conjugated lipid is PEG-DMG, and wherein the molar ratio of the cationic ionizable lipid, the helper lipid, the sterol, and the conjugated lipid is about 50%: 10%: 38.5%: 1.5% (mol) of the total lipid present in the LNP composition.

11. The LNP composition of any one of claims 1-10, wherein the LNP has a negative zeta potential.

12. The LNP composition of claim 11, wherein the LNP has a zeta potential between about −0.1 mV to about −15 mV, about −1.0 mV to about −10 mV, about −2.0 mV to about −8.0 mV, about −4.0 mV to about −6.0 mV, or about −5.0 mV to about −10 mV.

13. The LNP composition of claim 11, wherein the LNP has a zeta potential of at least-0.1 mV, at least-0.5 mV, at least-1.0 mV, at least-2.0 mV, at least-5.0 mV, at least-10 mV, or at least-15 mV.

14. The LNP composition of claim 11, wherein the LNP has a zeta potential of at most-0.1 mV, at most-0.5 mV, at most-1.0 mV, at most-2.0 mV, at most-5.0 mV, at most-10 mV, or at most-15 mV.

15. The LNP composition of any one of claims 1-14, wherein the LNP composition has a polydispersity index (PDI) of less than 1.0, less than 0.9, less than 0.8, less than 0.7, less than 0.6, less than 0.5, less than 0.4, less than 0.3, less than 0.2, or less than 0.1.

16. The LNP composition of any one of claims 1-15, wherein the LNP composition has a nitrogen/phosphate (N/P) ratio is about 2, about 3, about 4, about 5, about 6, about 7, about 8, about 9, about 10, or about 15.

17. The LNP composition of claim 16, wherein the N/P ratio is about 4.

18. A pharmaceutical composition, comprising the LNP composition of any one of claims 1-17, and a pharmaceutically acceptable carrier.

19. The pharmaceutical composition of claim 18, further comprising a cryoprotectant.

20. The pharmaceutical composition of claim 19, wherein the cryoprotectant is a sugar, starch, polyvinyl pyrrolidone, polyethylene oxide, or a combination thereof.

21. The pharmaceutical composition of any one of claims 18-20, wherein the pharmaceutical composition comprises a sugar.

22. The pharmaceutical composition of claim 21, wherein the sugar is a disaccharide.

23. The pharmaceutical composition of claim 20 or 21, wherein the sugar is selected from the group consisting of lactose, sucrose, trehalose, and a combination thereof.

24. The pharmaceutical composition of any one of claims 20-23, wherein amount of sugar is between about 0% w/v and about 40% w/v.

25. The pharmaceutical composition of any one of claims 20-24, wherein the amount of sugar is at least 5% w/v, at least 10% w/v, at least 20% w/v, or at least 30% w/v.

26. The pharmaceutical composition of any one of claims 20-25, wherein the amount of sugar is about 20% w/v.

27. A method of producing a pharmaceutical composition, comprising
a) producing a lipid nanoparticle (LNP) by mixing a lipid mixture and an aqueous solution, wherein
the lipid mixture comprises:
i. a cationic ionizable lipid;
ii. a helper lipid, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dimyristoyl-sn-glycero-phosphocholine (DMPC), and 1,2-dipalmitoyl-sn-glycero-3-phosphocholine (DPPC);
iii. a sterol, wherein the sterol is selected from the group consisting of cholesterol, β-sitosterol, campesterol, ergosterol, phytosterols (e.g., stigmastanol), and fucosterol;
iv. a conjugated lipid, wherein the conjugated lipid is selected from the group consisting of 1,2-dimyristoyl-sn-glycerol methoxypolyethylene glycol (PEG-DMG)-1000K, PEG-DMG-2000K, PEG-DMG-3000K, PEG-DMG-4000K and PEG-DMG-5000K; and
the aqueous solution comprises:
iv. a nucleic acid, wherein the nucleic acid is DNA and at least about 2 kilobases (kb), at least about 3 kb, at least about 4 kb, at least about 5 kb, at least about 6 kb, at least about 7 kb, at least about 8 kb, at least about 9 kb, or at least about 10 kb; and
b) mixing the LNPs produced in step a) and a pharmaceutically acceptable carrier.

28. The method of claim 27, wherein the cationic ionizable lipid is (6Z,9Z,28Z,31Z)-Heptatriaconta-6,9,28,31-tetraen-19-yl 4-(Dimethylamino)butanoate (DLin-MC3-DMA), or 2-(2,2-Di((9Z,12Z)-octadeca-9,12-dien-1-yl)-1,3-dioxolan-4-yl)-N,N-dimethylethanamine (DLin-KC2-DMA).

29. The method of claim 27 or 28, wherein the cationic ionizable lipid is DLin-MC3-DMA.

30. The method of any one of claims 27-29, wherein the cationic ionizable lipid is DLin-KC2-DMA.

31. The method of any one of claims 27-30, wherein the helper lipid is DOPC.

32. The method of any one of claims 27-31, wherein the sterol is cholesterol.

33. The method of any one of claims 27-32, wherein the pharmaceutical composition comprises a sugar.

34. The method of any one of claims 27-33, wherein the molar ratio of the cationic ionizable lipid, the helper lipid, the sterol, and the conjugated lipid is about 20%-80%: 5%-20%: 36%-39%: 1%-4% (mol) of the total lipid present in the LNP composition.

35. The method of any one of claims 27-34, wherein the molar ratio of the cationic ionizable lipid, the helper lipid, the sterol, and the conjugated lipid is about 50%: 10%: 38.5%: 1.5% (mol) of the total lipid present in the LNP composition.

36. The method of any one of claims 27-35, wherein the molar ratio of the cationic ionizable lipid, the helper lipid, the sterol, and the conjugated lipid is about 50%: 10%: 38.5%: 1.5% (mol) of the total lipid present in the LNP composition.

37. The method of any one of claims 27-36, wherein the cationic ionizable lipid is DLin-MC3-DMA, the helper lipid is DOPC, the sterol is cholesterol, and the conjugated lipid is PEG-DMG-2000K, and wherein the molar ratio of the cationic ionizable lipid, the helper lipid, the sterol, and the conjugated lipid is about 50%: 10%: 38.5%: 1.5% (mol) of the total lipid present in the LNP composition.

38. The method of any one of claims 27-36, wherein the cationic ionizable lipid is DLin-KC2-DMA, the helper lipid is DOPC, the sterol is cholesterol, and the conjugated lipid is PEG-DMG-2000K, and wherein the molar ratio of the cationic ionizable lipid, the helper lipid, the sterol, and the conjugated lipid is about 50%: 10%: 38.5%: 1.5% (mol) of the total lipid present in the LNP composition.

39. A method of treating a disease or disorder, the method comprising administering the LNP composition of any one of claims 1-17, the pharmaceutical composition of claim 18-26, or the pharmaceutical composition produced by the method of any one of claims 27-38 to a subject in need thereof.

40. The method of claim 39, wherein the disease or disorder is a lysosomal storage disease, an inborn errors of metabolism (IEM) or a hematological disorder.

41. The method of claim 39, wherein the LNP composition of any one of claims 1-17, the pharmaceutical composition of claim 18-26, or the pharmaceutical composition produced by the method of any one of claims 27-38 is administered intravenously.

42. The method of claim 39, wherein the LNP composition of any one of claims 1-17, the pharmaceutical composition of claim 18-26, or the pharmaceutical composition produced by the method of any one of claims 27-38 is administered subcutaneously or intramuscularly.

43. A method of optimizing Lipid Nanoparticle (LNP) comprising steps of (1) screening LNPs in vitro and (2) screening LNPs in vivo using immune deficient animal.

44. The method of claim 43, wherein the LNP comprises DNA.
